(19) United States
(12) Patent Application Publication
(10) Pub. No.: US 2014/0200626 A1
(43) Pub. Date: Jul. 17, 2014

(54) IMPLANTABLE TRANSIENT NERVE STIMULATION DEVICE

(71) Applicant: TRANSIENT ELECTRONICS, INC., Waltham, MA (US)

(72) Inventors: A. Stewart Campbell, Framingham, MA (US); Carmichael S. Roberts, JR., Brookline, MA (US); John A. Rogers, Champaign, IL (US); Christopher Poirier, Waltham, MA (US); Amy Manocchi, Waltham, MA (US); Daniel Harburg, Waltham, MA (US)

(73) Assignee: TRANSIENT ELECTRONICS, INC., Waltham, MA (US)

(21) Appl. No.: 14/154,938

(22) Filed: Jan. 14, 2014

Related U.S. Application Data
(60) Provisional application No. 61/912,731, filed on Dec. 6, 2013.
(60) Provisional application No. 61/753,122, filed on Jan. 16, 2013.
(60) Provisional application No. 61/752,717, filed on Jan. 15, 2013.

(51) Int. Cl. A61N 1/375; A61N 1/36

(52) U.S. Cl. CPC A61N 1/375; A61N 1/36071
USPC 607/46

(57) ABSTRACT

The invention generally relates to an implantable, tunable, and bioresorbable medical device for nerve stimulation within a body of a patient for pain management. The medical device includes a substrate, a circuit configured to provide stimulation to a target tissue, and a material surrounding the substrate and the circuit. The system further includes a controller configured to be disposed external to the patient's body and wirelessly communicate with the medical device to provide stimulation to the target tissue when the device is implanted within the patient's body. The substrate, circuit, and encapsulation layer may each include materials and/or have specific dimensions resulting in predictable and controllable resorption rates, such that the medical device may cease to function and completely dissipate within a medically relevant timescale (e.g., after completion of treatment).

### CROSS-REFERENCE TO RELATED APPLICATIONS

This application claims the benefit of, and priority to, U.S. Provisional Application Ser. No. 61/752,717, filed Jan. 15, 2013, U.S. Provisional Application Ser. No. 61/753,122, filed Jan. 16, 2013, and U.S. Provisional Application Ser. No. 61/912,731, filed Dec. 6, 2013, the contents of which are incorporated by reference herein in their entirety.

### FIELD

The present disclosure relates generally to transient devices, and, more particularly, to an implantable, tunable, and bioresorbable medical device for nerve stimulation within a body of a patient for pain management.

### BACKGROUND

Pain management is widely recognized as a major medical challenge. This is particularly true in the military field, where chronic neuropathic pain management persists as one of the most ongoing and significant medical challenges, impacting the full spectrum of military personnel, including in the active duty, Wounded Warrior, Warrior in Transition (WIT), and Veteran populations. Pain is the single most prevalent driver for current and former military personnel to seek medical attention. In fact, the majority of the 42,000 daily MEDCOM visits and the 5.8 million annual VHA visits involve a pain assessment, wherein pain is often referred to among medical staff as the “5^{th} Vital Sign”.

Chronic pain is typically classified as pain lasting more than 6 months and generally divided into three main types: nociceptive, psychogenic or neuropathic (e.g., due to nerve injury) although the distinction between these types can be blurred. Current approaches to the management of chronic pain include pharmacological and Complementary Alternative Medicine (CAM) strategies. Opiates and analgesics are the most often prescribed pharmacological agents, and while they are usually effective at relieving pain symptoms, the use of these agents is fraught with problematic side effects and drawbacks including addiction and/or motor function and gastrointestinal side effects. Such side effects can hamper soldier recovery and rehabilitation and can promote “passive patient mentality” in which the soldier becomes focused primarily on receiving treatment for pain and less on being an active participant in their own recovery. CAM techniques include acupuncture, yoga, massage, and the use of electrical nerve stimulation. Currently, these techniques are used to augment or supplement the use of opiates and analgesics, and have yet to emerge as primary pain treatment methods.

While electrical stimulation has been shown to facilitate several biophysical processes, including wound healing, enhancement of muscular strength, and bone growth, its use in pain relief has been most widely investigated. Electrical stimulation of peripheral nerves or the spinal cord directly can yield therapeutic benefit if applied properly. However, state-of-the-art devices are suboptimal. State-of-the-art devices for nerve stimulation to treat pain typically utilize either transcutaneous electrical nerve stimulation (TENS) or percutaneous electrical nerve stimulation (PENS), both of which suffer from drawbacks that limit their widespread use.

TENS is a non-invasive technique in which all components are external with the electrodes placed on the skin of the patient. Applied current ranges from <2 mA (low-intensity) to <15 mA (high-intensity) delivered at either low frequency (<10 Hz) or high frequency (50-150 Hz). The “high-frequency” signals of the TENS technique, however, fall well-short of the 1000 Hz frequency generally required for deep tissue penetration, resulting in the applied current traveling between the electrodes along the surface of or just beneath the skin. Mechanistically, TENS is thought to work through stimulation of small-diameter cutaneous nerve fibers at the site of application, leading to the common practice of placing external electrodes at or near the site of injury. However, recent studies indicate that both peripheral and central mechanisms that rely on engagement of large-diameter afferent nerve fibers are likely operative. Central mechanisms are far more biologically complex, involving stimulation of a wide range of opioid receptors and multiple fibers within a network. The determination of which receptors are stimulated appears to depend on the applied signal (frequency, current) and the composition and location of the nerve fiber itself. This evolving understanding of the mechanisms by which TENS may achieve analgesia may partially explain the historically mixed clinical results, and hence controversial status of TENS within the medical community. Thus, the ideal operative parameters for TENS devices in a given pain management situation remain unclear. Accordingly, TENS in its current form is likely to remain a secondary intervention approach.

PENS is an invasive technique in which the stimulating electrodes are implanted near the affected site or the spinal cord. The applied electrical signal is generated either by an implanted power source or via epidermal capacitive coupling or non-contact microwave transmission to create an electrical field that stimulates afferent neurons or the spinal cord directly. In the event that the correct stimulatory signal is generated in the correct dimension at the correct position, the result is paresthesia, a sensation of tingling, tickling, prickling, pricking, or burning that may mask the pain.

Since the introduction of PENS as a pain management technique, improvements in clinical outcomes have resulted from refined surgical procedures, improved equipment and optimized stimulation programs. PENS is considered by some to be the most promising paradigm for clinically relevant nerve stimulation or percutaneous neuromodulation to treat a variety of pain-related indications. However, despite this promise, significant technical challenges and drawbacks exist, limiting a broader use of PENS. For example, lead breakage and migration are major complications with PENS devices. Up to 30% of patients experience treatment disruptions or suboptimal device function. PENS devices are associated with increased risk of infection and require repeated surgeries to retrieve or replace the electrodes. Improper placement of electrodes can lead to perineural scarring and fibrosis, which can lead to restricted nerve function when administered over long treatment periods. The need for subsequent surgeries to repair, replace or remove PENS devices is a major drawback.

### SUMMARY

The present invention provides systems and method for treating pain conditions. In one aspect, a system includes an implantable, biocompatible, tunable, and bioresorbable medical device for peripheral nerve stimulation for the management of pain. The medical device includes a substrate, a circuit configured to provide stimulation to one or more nerve fibers, and a material surrounding the substrate and the circuit. The system further includes a controller configured to be disposed external to the patient's body and wirelessly communicate with the medical device to provide stimulation to the target tissue when the device is implanted within the patient's body.

The circuit of the medical device includes electronic components, which may form an integrated circuit, including, but not limited to, conducting electrodes and interconnects, dielectrics, and semiconductor material, all supported by the substrate. In some embodiments, one or more of the electronic components of circuit and the supporting substrate are bioresorbable (e.g., able to degrade and break down when implanted into the body of a patient) and are also biocompatible, such that degraded components do not cause toxicity and/or inflammation. The circuit and substrate are further encapsulated by a protective bioresorbable layer so as to enable implantation within the patient. The substrate, circuit, and encapsulation layer may each include materials and/or have specific dimensions or geometries resulting in predictable and controllable resorption rates, such that the medical device may cease to function and completely dissipate within a medically relevant timescale (e.g., after completion of treatment).

The medical device may be implanted subcutaneously at or in close proximity to a trauma site, such that a stimulatory signal from the medical device will reach and address the relevant afferent neurons of a nerve fiber of interest, although direct contact between the electrodes and a nerve fiber is not necessary. The medical device may be immobilized at the time of transplantation by way of bioresorbable fixtures, such as sutures or staples. In one embodiment, the fixtures are configured to degrade at the same rate as the implanted medical device. In another embodiment, the fixtures may provide temporary immobilization until the medical device is fixed within the implant site via immunologically-driven encapsulation by fibrous extracellular matrix material. In another embodiment, the circuit and substrate may be sufficiently flexible such that the medical device may be configured to physically conform to the implant site and/or target nerves, thus precluding the requirement for immobilization.

Nerve stimulation to relieve pain is achieved by wirelessly transmitting high frequency signals from the external controller to the medical device. Upon receiving high frequency signals, a current flows between the electrodes of the circuit, wherein the electrodes are configured to deliver electrical energy to the one or more nerve fibers to stimulate paresthesia, thereby masking associated pain. In particular, the electrodes are configured to generate an electric field that penetrates surrounding tissue containing the affected sensory or peripheral nerves. The electrodes are configured to deliver a variety of different stimulation patterns based on wireless input from the external controller. For example, the external controller may operate in a variety of different modes, each mode resulting in the delivery of a different stimulation pattern from the electrodes. Accordingly, the system allows the tuning of stimulation patterns on a patient-by-patient basis for frequency, amplitude and duration so as to inhibit the transmission of pain signals along the nerve fibers, thereby providing pain relief.

The implantable transient nerve stimulation device of the present invention provides numerous benefits. For example, most, if not all, of the components of the implantable medical device are composed of materials having predictable and controllable resorption within a patient upon implantation. The bioresorbable characteristics of the medical device circumvent the technical limitations of current nerve stimulation devices and methods, such as TENS and PENS devices. For example, the target duration of operation of the device may be a function of the expected period of treatment. The transience of function may be controlled either by incorporating one or more bioresorbable components within the circuit of the device itself or by including a bioresorbable protective encapsulation coating configured to degrade over a programmed period of time, after which the circuit is compromised and ceases function. Once the functional phase of the device is terminated, the remnants of the implanted device may be resorbed naturally over a much longer time period. Accordingly, the medical device of the present invention may degrade after a desired period of time (e.g., upon completion of treatment), further eliminating the need for repeated surgeries and risk of infection or inconvenience to the patient.

Furthermore, the medical device has wireless capabilities, such that an external controller may be used to both wirelessly transmit power to and control output (e.g., stimulation) from the device. The use of wireless communication overcomes the drawbacks associated with devices having wired connections. For example, some implantable devices must be directly connected to an external power source or controller in order to function, wherein, in addition to being inconvenient to a patient, the wire connecting the external power source or controller and the device must be constantly cleaned and monitored to avoid infection. The ability to wirelessly control of the medical device of the present invention overcomes the drawbacks associated with wired connections, thus improving patient treatment and compliance.

Additionally, the transient medical device includes optimal bioresorbable materials and manufacturing processes, allowing the medical device to achieve electronic performance profiles closely comparable to those of non-transient or resorbable implantable devices based on conventional silicon-on-insulator (SOI) electronics. SOI-based flexible electronic devices consistently have shown superior reliability, durability and performance versus organic material-based microelectronic devices. Silicon based approaches, unlike organics, are well-aligned with a large, existing industry and benefit, as a result, from an established base of engineering and technical knowledge in device and circuit design for reliability and performance. Modern silicon electronics, such as SOI electronics, do not provide transient capabilities. Accordingly, the transient medical device is configured to provide comparable SOI-like electronic performance, while still being bioresorbable on a medically relevant timescale, thus exploiting the benefits of modern silicon electronics and the benefits of transient technology.

The devices specifically proposed herein are intended to treat subjects suffering from sub-chronic and chronic pain. In one aspect, the devices are configured to be used to treat military personnel, for example, such that the devise may be utilized by forward surgical teams or aid stations and in combat support hospitals. For the purposes of discussion, the following description focuses on a device for the treatment of somatic and visceral nociceptive pain associated with battlefield polytrauma, burns, lacerations and post-surgical pain. However, it should be noted that systems and methods described herein may be used for treating and managing other types of pain and/or in connection with the general population (i.e. civilians).

### BRIEF DESCRIPTION OF THE DRAWINGS

Features and advantages of the claimed subject matter will be apparent from the following detailed description of embodiments consistent therewith, which description should be considered with reference to the accompanying drawings, wherein:

FIG. 1 is a block diagram illustrating one embodiment of an exemplary system for stimulating a target tissue within a body of a patient consistent with the present disclosure.

FIG. 2 is a top plan view of one embodiment of an implantable transient medical device of the system of FIG. 1.

FIG. 3 is a cross-sectional view of the implantable transient medical device of FIG. 2.

FIG. 4 is a top plan view of a patterned trace material for use in an implantable transient medical device consistent with the present disclosure.

FIG. 5 is a perspective view of the patterned trace material of FIG. 4 disposed on a substrate and coupling one or more components to one another to form the circuit of the medical device.

FIG. 6 is an image depicting a completed circuit, including components coupled to one another by the trace material and disposed on the substrate of FIG. 5.

FIGS. 7A and 7B are graphs illustrating dissolution properties of one exemplary bioresorbable substrate material.

FIGS. 8A-8C are images depicting the appearance of the exemplary bioresorbable substrate material of FIGS. 7A and 7B upon wetting.

FIGS. 9A and 9B are graphs illustrating dissolution properties of the exemplary bioresorbable substrate material of FIGS. 7A and 7B.

FIGS. 10A and 10B are graphs illustrating degradation and water adsorption profiles of another exemplary bioresorbable substrate material.

FIGS. 11A and 11B are graphs illustrating dissolution properties and degradation/water adsorption profiles of another exemplary bioresorbable substrate material.

FIGS. 12A-12F are graphs illustrating resistance changes during the dissolution of different exemplary bioresorbable metals for use as one or more components in the circuit of a transient medical device consistent with the present disclosure.

FIG. 13 illustrates one embodiment of a circuit of the transient medical device of the system of FIG. 1 consistent with the present disclosure.

FIG. 14 is a graph illustrating exemplary circuit input and circuit output for peripheral nerve stimulation.

FIG. 15 illustrates another embodiment of a circuit of the external controller and transient medical device of the system of FIG. 1 consistent with the present disclosure.

FIG. 16 illustrates another embodiment of a circuit of the external controller and transient medical device of the system of FIG. 1 consistent with the present disclosure.

FIG. 17 is a graph illustrating different voltages observed in the circuit of FIG. 16 upon simulation of the circuit.

FIGS. 18A and 18B are perspective views of an exemplary external controller (e.g., transmitter) wirelessly communicating with an exemplary transient medical device (e.g., receiver) through different mediums (air in FIG. 18A and saline solution in FIG. 18B).

FIG. 19 is a graph illustrating different voltages observed during operation of the systems of FIGS. 18A and 18B.

FIG. 20 illustrates another embodiment of a circuit of the external controller and transient medical device of the system of FIG. 1 consistent with the present disclosure.

FIG. 21 is a graph illustrating a range of tolerant stimulation levels configured to be delivered by the circuitry of the transient medical device of FIG. 20.

FIG. 22 is a sectional anterior view of a portion of a patient's torso, illustrating the implantation of a transient medical device consistent with the present disclosure adjacent to the rectus femoris muscle of the leg.

FIG. 23 is an enlarged view, partly in section, of the rectus femoris muscle including a bundle of peripheral nerves targeted with the electrical field generated and delivered from the transient medical device.

FIG. 24 is a flow diagram illustrating one embodiment of a method for stimulating a target tissue within a body of a patient.

For a thorough understanding of the present disclosure, reference should be made to the following detailed description, including the appended claims, in connection with the above-described drawings. Although the present disclosure is described in connection with exemplary embodiments, the disclosure is not intended to be limited to the specific forms set forth herein. It is understood that various omissions and substitutions of equivalents are contemplated as circumstances may suggest or render expedient.

### DETAILED DESCRIPTION

By way of overview, the present disclosure is generally directed to systems and method for treating pain. For the purposes of discussion, the following description focuses on systems and methods for treating sub-chronic and/or chronic pain in military personnel, particularly treatment of somatic and visceral nociceptive pain associated with battlefield polytrauma, burns, lacerations and post-surgical pain. However, it should be noted that systems and methods described herein may be used for pain treatment and management in general population (i.e. civilians).

In one aspect, a system includes an implantable, biocompatible, tunable, and bioresorbable medical device for peripheral nerve stimulation for the management of pain. The medical device includes a substrate, a circuit configured to provide stimulation to one or more nerve fibers, and a material surrounding the substrate and the circuit. The system further includes a controller configured to be disposed external to the patient's body and wirelessly communicate with the medical device to provide stimulation to the target tissue when the device is implanted within the patient's body.

The circuit of the medical device includes electronic components, which may form an integrated circuit, including, but not limited to, conducting electrodes and interconnects, dielectrics, and semiconductor material, all supported by the substrate. In some embodiments, one or more of the electronic components of circuit and the supporting substrate are bioresorbable (e.g., able to degrade and break down when implanted into the body of a patient) and are also biocompatible, such that degraded components do not cause toxicity and/or inflammation. The circuit and substrate are further encapsulated by a protective bioresorbable layer so as to enable implantation within the patient. The substrate, circuit, and encapsulation layer may each include materials and/or have specific dimensions resulting in predictable and controllable resorption rates, such that the medical device may cease to function and completely dissipate within a medically relevant timescale (e.g., after completion of treatment).

Nerve stimulation to relieve pain is achieved by wirelessly transmitting high frequency signals from the external controller to the implanted medical device. Upon receiving high frequency signals, a current flows between the electrodes of the circuit, wherein the electrodes are configured to deliver electrical energy to the one or more nerve fibers to stimulate paresthesia, thereby masking associated pain. The system further provides tuning of stimulation patterns, such as adjustment of frequency, amplitude, and/or duration, thereby allowing customization of pain treatment on a patient-by-patient basis.

Most, if not all, of the components of the implantable medical device are composed of materials having predictable and controllable resorption within a patient upon implantation. Accordingly, the target duration of the function life of the device may be a function of the expected period of treatment. The transience of function may be controlled either by incorporating one or more bioresorbable components within the circuit of the device itself or by including a bioresorbable protective encapsulation coating configured to degrade over a programmed period of time, after which the circuit is compromised and ceases function. Once the functional phase of the device is terminated, the remnants of the implanted device may be resorbed naturally over a much longer time period. Accordingly, the medical device of the present invention may degrade after a desired period of time (e.g., upon completion of treatment), further eliminating the need for repeated surgeries and risk of infection or inconvenience to the patient.

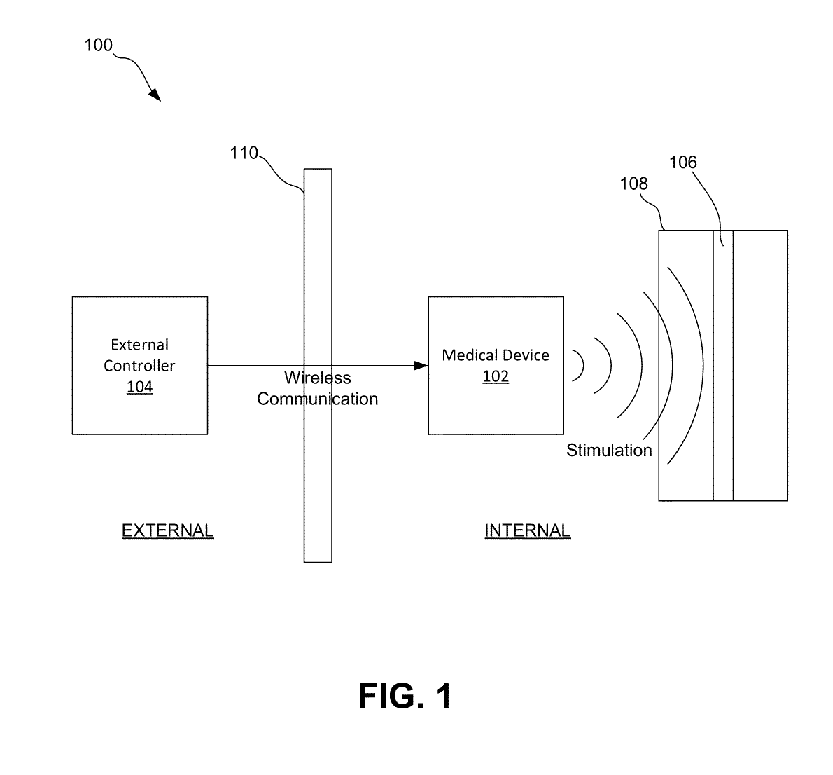

Turning to FIG. 1, one embodiment of an exemplary system 100 for stimulating a target tissue within a body of a patient is generally illustrated. As shown, the system 100 includes a medical device 102 implanted within a patient's body 110 (e.g., internally) and a controller 104 disposed external to the patient's body 110 and configured to wirelessly communicate with the medical device 102. Upon to receiving wireless input from the controller 104, the medical device 102 is configured to provide stimulation to a target tissue 106. The target tissue 106 may include, but is not limited to, heart tissue, brain tissue, muscle tissue, epithelial tissue, nerve tissue, and vascular tissue. As shown, the stimulation delivered from the medical device 102 is configured to penetrate surrounding tissue 108 and reach the target tissue 106. For example, the target tissue includes one or more nerve fibers 106 surrounded by muscle tissue 108. The stimulation provided by the medical device 102 includes electrical energy configured to stimulate paresthesia, for example, within the one or more nerve fibers 106 so as to treat and manage pain associated with the nerve fibers 106, as described in greater detail herein.

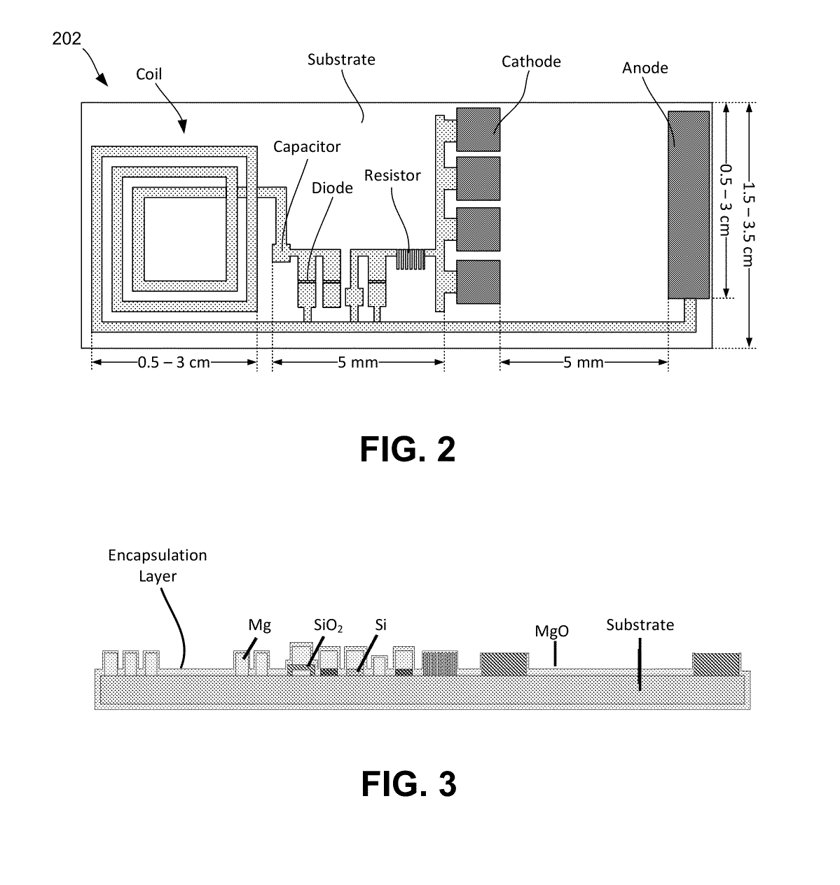

FIG. 2 is a top plan view of one embodiment of an implantable transient medical device—202 of the system 100 of FIG. 1 and FIG. 3 is a cross-sectional view of the implantable transient medical device 202. As shown, the medical device 202 generally includes a substrate, a circuit configured to provide stimulation to one or more nerve fibers, and a material surrounding the substrate and the circuit (e.g., encapsulation layer). The circuit of the medical device 202 includes electronic components, including, but not limited to, conducting electrodes and interconnects, dielectrics, and semiconductor components, all supported by the substrate. In some embodiments, one or more of the electronic components of circuit and the supporting substrate are biodegradable and/or bioresorbable, as well as biocompatible, such that degraded components do not cause toxicity and/or inflammation if degraded within a patient's body. The circuit and substrate are further encapsulated by a protective bioresorbable layer so as to enable implantation within the patient.

The term “biodegradable” generally refers to a material that has a chemical structure that may be altered and is susceptible to being chemically broken down into lower molecular weight chemical moieties by common environmental chemistries (e.g., enzymes, pH, and naturally-occurring compounds) to yield elements or simple chemical structures that may be resorbed by the environment. The term “bioresorbable” generally refers to a material that is susceptible to being chemically broken down into lower molecular weight chemical moieties by chemical and/or physical process upon interaction with one or more components (e.g., reagents) in a physiological environment, such as a within the body of a human or animal. The material may be broken down into components that are metabolizable or excretable. For example, in an in-vivo application, the chemical moieties may be assimilated into human or animal tissue. The term “biocompatible” refers to a material that does not elicit an immunological rejection or detrimental effect when it is disposed within an in-vivo biological environment. For example, a biological marker indicative of an immune response changes less than 10%, or less than 20%, or less than 25%, or less than 40%, or less than 50% from a baseline value when a biocompatible material is implanted into a human or animal.

As shown, the circuit of the medical device 202 includes a trace pattern forming an inductive coil, one or more capacitors, one or more resistors, and contact pads for connecting semiconductor devices, as well as electrodes, to the circuit. The medical device 202 is configured to wirelessly receive input from the external controller 104 via the inductive coil of the circuit, and, in turn, the electrodes are configured to output electrical energy. The particular arrangement and configuration of the circuit is configured to adjust one or more properties of electrical energy delivered from the electrodes to the target tissue, as described in greater detail herein. As shown, the overall dimensions of the medical device 202 are 5 cm² or less, thereby allowing the medical device 202 to be easily implanted and positioned within a variety of different target sites.

As previously described, the substrate, circuit, and encapsulation layer may each include materials and/or have specific dimensions resulting in predictable and controllable resorption rates, such that the medical device 202 may cease to function and completely dissipate within a medically relevant timescale (e.g., after completion of treatment). For example, as shown in FIG. 3, one or more components of the circuit comprises a material selected from the group consisting of magnesium (Mg), Mg alloys, magnesium oxide (MgO), zinc (Zn), tungsten (W), iron (Fe), silicon (Si), silicon oxide (SiO₂), and combinations thereof. As shown, Mg is used to fabricate coils, contact pads, capacitors, and resistors, while diodes are fabricated with silicon derived from a silicon-on-insulator (SOI). For example, in one embodiment of a fabrication method, Mg foils are patterned using a laser-cutting method and transfer printed from adhesive tape to the substrate.

In one embodiment, ultrathin single crystalline silicon nanomembranes (SiNMs) may serve as the semiconductor material for proposed transient electronic devices. There is strong rationale and precedent for utilizing SiNMs as the semiconductor component, including SOI-level carrier mobility (e.g. 560 cm 2/V-s (saturation mobility), 660 cm 2/V-s (linear regime mobility) for proof of concept n-channel devices), practical fabrication via photolithography and reactive-ion etching (SF 6 gas) of SOI wafers followed by a wet etch release of the SiNMs and finally transfer printing onto the device substrate, and a controlled aqueous dissolution profile on the time scale of weeks based on the SiNM thickness, a period consistent with the target transience period.

Silicon oxide (SiO₂) and magnesium oxide (MgO) may further be used as interlayer dielectrics in the circuit of the medical device 202. While SiO₂ may be a preferred material for use in integrated circuits (ICs) due to their performance, versatility and reliability, both metal oxides are compatible with conventional fabrication conditions and techniques, including the temperature and pressure extremes of e-beam and CVD, which can produce high-purity, high performance, homogeneous interlayer dielectrics. These materials also may be deposited on virtually any type of underlying substrate material. Notably, MgO also has the benefit of acting as an adhesion promoter for metal conductors. Furthermore, ultrathin SiO₂ and MgO dissolve in aqueous solution on a time scale similar to that of SiNMs.

Accordingly, SiO₂ may be deposited as a dielectric material that is sandwiched between the parallel capacitive plates and the crossover regions of the coil. For example, doped monocrystalline silicon nanomembrane (SiNM,—300 nm thick) semiconductors prepared from SOI wafers by high temperature diffusion of phosphorus and boron into defined regions. Isolation of the SiNMs can be achieved by reactive-ion etching (RIE) using sulfur hexafluoride (SF₆) gas. The SiNMs are released from the wafer by wet etching with aqueous HF, and transfer printed onto a substrate material. Stencil masks are used to enable patterned deposition of the metal electrodes, dielectrics and interconnects (if needed), for example via e-beam evaporation or chemical vapor deposition.

In the illustrated embodiment, the cathodes consist of arrays on electrodes that are distributed equidistant along affected nerve sites, while the anode usually contains one electrode that is posited on the surrounding soft tissues or the adjacent area of the cathodes. The electrodes and interconnects are made of conductive materials, such as Mg, Mg alloys, and W, in this particular example.

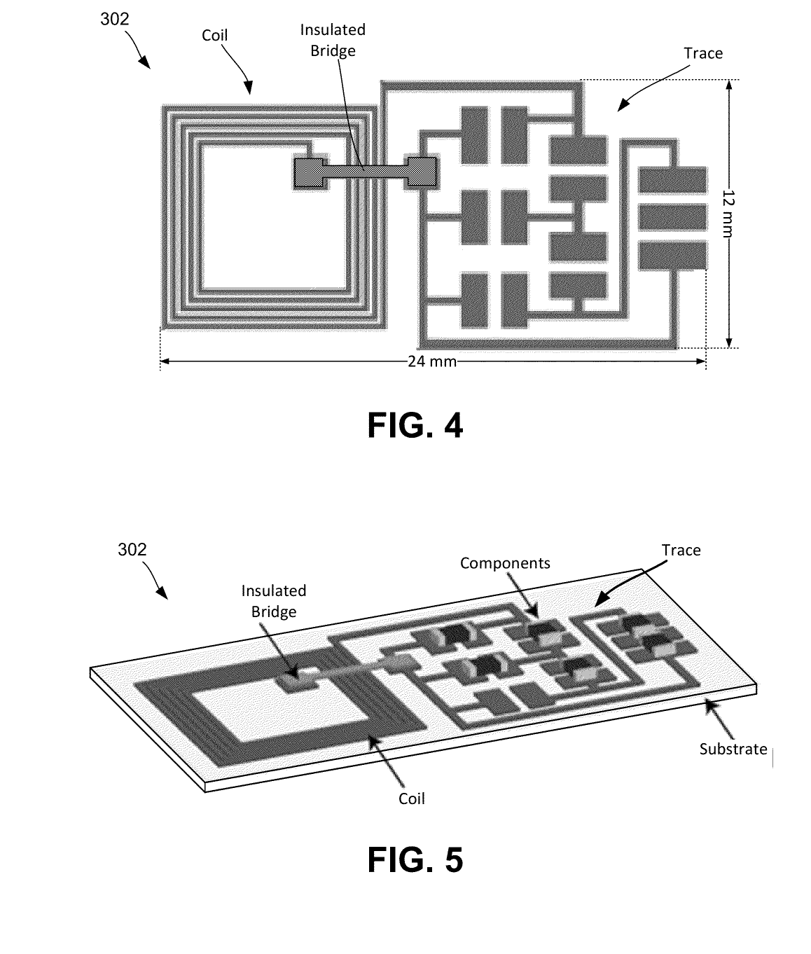

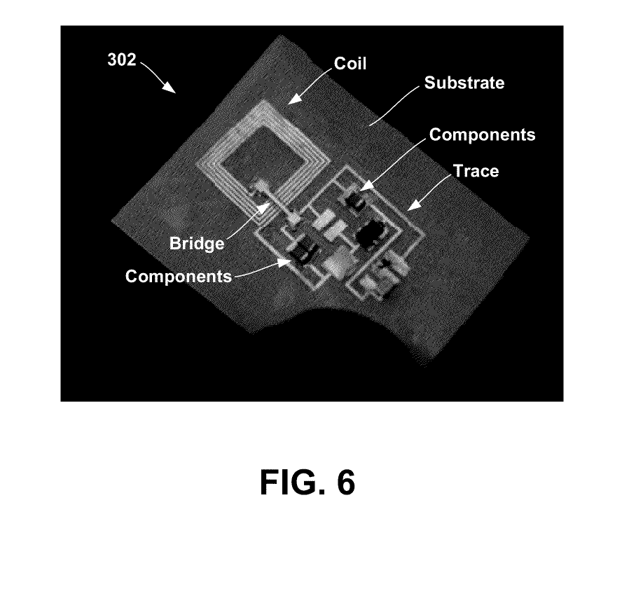

FIG. 4 is a top plan view of a patterned trace material for use in an implantable transient medical device 302 consistent with the present disclosure. FIG. 5 is a perspective view of the patterned trace material of FIG. 4 disposed on a substrate and coupling one or more components to one another to form the circuit of the medical device 302. FIG. 6 is an image depicting a completed circuit, including components coupled to one another by the trace material and disposed on the substrate of FIG. 5. The medical device 302 of FIGS. 4-6 was fabricated similarly of the device 202 of FIGS. 3 and 4, including similar materials. Generally, fabrication of the medical device 302 requires three fabrication steps: patterning of the magnesium traces, transfer printing of the magnesium to the transient substrate, and bonding of the components to the magnesium traces. The Mg foils can be used to form inductive coils, capacitors, and traces connecting semiconductor devices on transient substrates. In the illustrated embodiment, the Mg foils have a thickness of 60 μm. However, it should be noted that the Mg foils may have a greater or lesser thickness depending on the desired AC resistance characteristics. As such, the Mg foils may have a thickness ranging between 10 and 100 μm, 1 and 1000 μm, etc.

As shown in FIGS. 5 and 6, Mg foils were patterned and transfer printed from adhesive tape to a degradable substrate. A thin surface layer of the substrate was dissolved in chloroform to adhere the Mg traces to the substrate. Once the solvent fully evaporated, the adhesive tape was peeled away leaving the Mg pattern behind. Further, a bridge structure was formed using insulated Mg foil to connect the inner terminal of the receiver coil to the common ground node of the receiver. The bridge structure may comprise one or more flexible and/or stretchable electrical interconnections providing electrical communication between elements. Next, surface-mount components were then bonded to the traces using conductive silver paint, rendering these circuits partially transient.

The design of safe, functional, implantable, transient medical device requires that the mechanical, chemical, physicochemical, and biological properties of the substrate and encapsulation materials are carefully considered. The specific properties of a given material can impact device fabrication, storage, handling, deployment and, critically, the transience profile. Moreover, certain properties that may facilitate fabrication, for example, may have adverse effects on the transience profile. As described in greater detail herein, studies were performed in determining the most promising substrate and/or encapsulation material for an implantable transient medical device consistent with the present disclosure. The results of the study indicate that the substrate and/or encapsulation layer comprises a biodegradable and/or bioresorbable material selected from the group consisting of polyanhydrides, polyortho-esters, polyesters, polyphosphazenes, and combinations thereof. The circuit and substrate are then encapsulated with a thin insulating layer of transient material to allow time-controlled interface with interstitial fluids upon implantation.

In the medical devices of the present invention, it is crucial to select proper substrate and/or encapsulation materials having certain properties, so as to allow predictable degradation in a medical relevant timescale, while still providing sufficient support and function for circuitry of the transient medical device. Substrate materials must retain sufficient robustness and mechanical stability (e.g. modulus >10 MPa) to support the electronics and accommodate the device fabrication sequences; however, they must also be flexible enough (modulus <10 GPa) to enable casting of films and coatings for integration into biological tissues. Good tensile strength is required to withstand fabrication processes and the physical assaults on the device post-implantation.

The physicochemical properties of a material impact fabrication, function, and transience of the medical device. Fabrication sequences often require vacuum processing (e.g. e-beam evaporation and CVD); therefore the materials must have low vapor pressure, which for organic polymers can be a function of the amount of residual monomer present within the material. High glass transition temperature (T g) (e.g., T g of 100° C.), and high melt temperature (T m) ensure structural integrity of the materials during metal and dielectric deposition processes. For example, during electronic operations, a T g well above 37° C. is especially important for implantable electronic medical devices, since a functioning device will reach temperatures above body temperature during operation. Additionally, it may be desirable that materials have a very low T g (e.g., T g below 4° C.), such that, if the T g is very low and the T m is high, there will be no physical transition event within the relevant temperature range, either during fabrication or during electronic operation of the medical device.

In this regard, materials with high thermal conductivity that are able to dissipate heat may be especially attractive. The transience profile of a device is heavily influenced by the hydrophilic/hydrophobic nature and intrinsic solubility of its component materials. Hydrophilic materials of low crystallinity (e.g. PEG) will absorb water and swell, can induce the fracture or delamination of metal or semiconductor patterns on polymer films. Swelling may be reduced by increasing the hydrophobicity and/or the crystallinity of the material, leading to more stable devices; however, some degree of water solubility is often desirable if control of the transience profiled is desired.

The most important properties for transient electronic devices are the chemical and enzymatic stability of the substrate and encapsulation materials under physiological conditions and the mechanisms of degradation. The ability to optimize and control these properties dictates the functional time course and ultimately the success or failure of a particular device. The rate of material dissolution is a function of both the intrinsic chemical or enzymatic reaction rates and the interfacial surface area between the device material and its corrosive surroundings.

Depending on the physicochemical properties of the material, degradation may occur either by bulk erosion, or surface erosion. In bulk erosion, the rate of covalent bond scission through hydrolytic or enzymatic processes is slower that the rate at which the aqueous medium penetrates the material matrix. In the case of polymer materials, swelling occurs faster than the degradation process, and as described above, can lead to premature failure of a device.
Materials that degrade by bulk erosion therefore may not be best-suited for substrate or encapsulation materials for transient electronic devices. However, it is conceivable that a material prone to bulk erosion could be integrated into a device as a transience trigger.

In contrast to bulk erosion, surface erosion is the dominant process when the rate of hydrolysis or enzymatic degradation is faster than the rate of penetration of the material by the aqueous medium. By inhibiting water from diffusing into the material and displaying relatively reactive labile functional groups on the surface, the material will shrink over time through surface depletion at the surface. In the case of organic polymers, controlling the hydrophobicity and crystallinity of the material can be an effective means for limiting the degradation profile to surface erosion processes. For the proposed transient devices, it will be critical to identify organic and inorganic substrate materials that degrade primarily via surface erosion.

In addition to controlling the erosion profile, it is important that the surface of any substrate material be chemically modifiable to ensure good bonding to deposited or transfer printed materials (e.g. semiconductor or conductive material), thereby enabling fabrication of stable, functional devices.

Additionally, substrate and/or encapsulation materials should not invoke a strong inflammatory or toxic response upon implantation or upon degradation/resorption. Degradation products, whether resulting from hydrolysis or metabolism should either be completely metabolized or excreted via normal pathways in the body.

The mechanical, physicochemical, chemical and biological properties of substrate and/or encapsulation materials were studied and considered for their impact on functional potential, transience potential and compatibility with foundry fabrication sequences of the implantable transient medical device of the present invention. Four classes of materials were investigated due to their widely understood hydrolytic properties, and biocompatibility: poly(anhydrides), poly(ortho-esters), poly(esters) and poly(phosphazenes). The selection criteria included biocompatibility, hydrolytic degradability, surface degradability and controllable physical properties. Four materials were identified as candidates for experimental evaluation, Poly(thiol-ene) (PTE), Poly(caprolactone) (PCL), Poly(ortho-ester) (POE), and Poly(glycerol-sebacate) (PGS), as shown below:

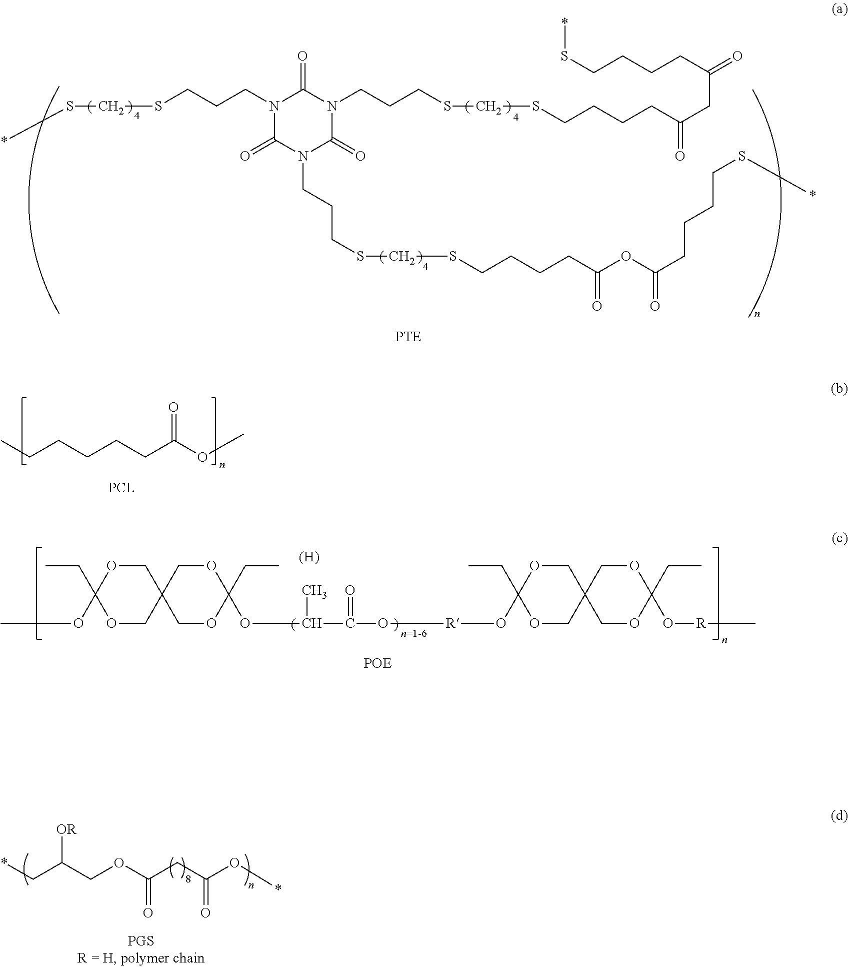

Poly(anhydrides), synthesized via thiol-ene chemistry, are easily prepared in a solvent-free system via UV polymerization, enabling facile synthesis and 3-D polymer structure flexibility. The wide range of commercially available monomers facilitates the simple tunability of material properties and degradation. Many PTE materials have been studied in drug delivery applications. PCL is a polyester material that has been heavily studied in implantable devices, and approved by the FDA. It has been fully characterized in the literature, and many forms are commercially available in large quantities. A class of POE was selected based on their widely published surface erosion characteristics and biocompatibility. Specifically, the material is based on the monomer “DETOSU” (3,9 diethylidene-2,4,8,10-tetraoxaspiro[5.5]undecane) and two linker molecules (trans cyclohexanedimethanol (tCHD) and hexanediol (HD)). The POE formulation of poly(DETOSU-tCHD-HD) (100:50:50) was selected as the benchmark material of this class, because of its well-reported and promising properties. PGS has well reported biocompatibility and elastomeric properties. PGS, a thermoset polyester, has been used extensively in implantable applications such as drug delivery and artificial tissue applications. The benchmark formulation was poly(glycerol-sebacic acid) (50:50), as it has been well studied and reported in the literature.

The four candidate materials (PTE, PCL, POE, and PGS) were prepared and analyzed to determine their suitability as substrate materials for the transient medical device of the present invention. Table 1, shown below, provides the experimental analysis of material dissolution and physical properties, thereby leading to at least three candidate substrate materials.

TABLE 1
Substrate material candidate dissolution properties

| | | Water Uptake at | Degradation | | |
|---|---|---|---|---|---|
| | Contact | 25° C. at pH 7.4 | at 25° C. at pH | Tensile | Lead |
| Polymer | Angle | in 7 days | 7.4 | Stress | Candidate |
| PTE (1:1) | 68.4° | 20 mg | 1.15 mg/day | * | No |
| PTE (1:2) | 71.8° | 0.4 mg | 0.3 mg/day | * | No |
| PTE (1:4) | 89.1° | 0.3 mg | 0.05 mg/day | 4.9 MPa | Yes |
| PCL80 | 69.2° | <0.1 mg | 0.15 mg/day | 9.2 MPa | Yes |
| PCL45 | 66.2° | <0.1 mg | <0.05 mg/day | 6.4 MPa | Yes |
| PGS (1:1) | 82.6° | 10 mg | 0.46 mg/day | 1.55 MPa | No |

Polyanhydrides were synthesized through thiol-ene chemistry by simple mixing of commercially available monomers, followed by UV polymerization. The properties were easily tailored by modifying the type of linker molecules and their relative ratios, and were rendered degradable by using a linker molecule possessing a hydrolysable anhydride functional group. PTE's were synthesized using various multi-armed divinyl linkers, different lengths of linear dithiols and the 4-pentenoic-anhydride (the degradable group), then screened for transience potential (water penetration and dissolution profiles) and suitability of physical properties. The best linker combination was selected for further investigation.

As shown below, the PTE selected was synthesized from 4-pentenoic-anhydride (4PA), a branched linker 1,3,5-Triallyl-1,3,5-triazine-2,4,6(1H,3H,5H)-trione (TTT) and a linear dithiol 1,4 butanedithiol (BDT). The system was modified by varying linker ratios; provided there were stoichiometrically equivalent vinyl and thiol groups. Four ratios of this PTE were considered material. As shown in Table 1, the contact angle measurements show that hydrophobicity increases with increasing amount of BDT.

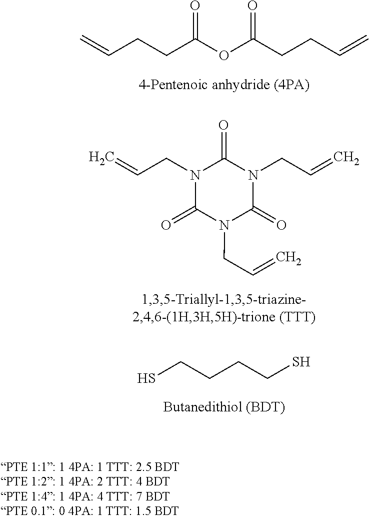

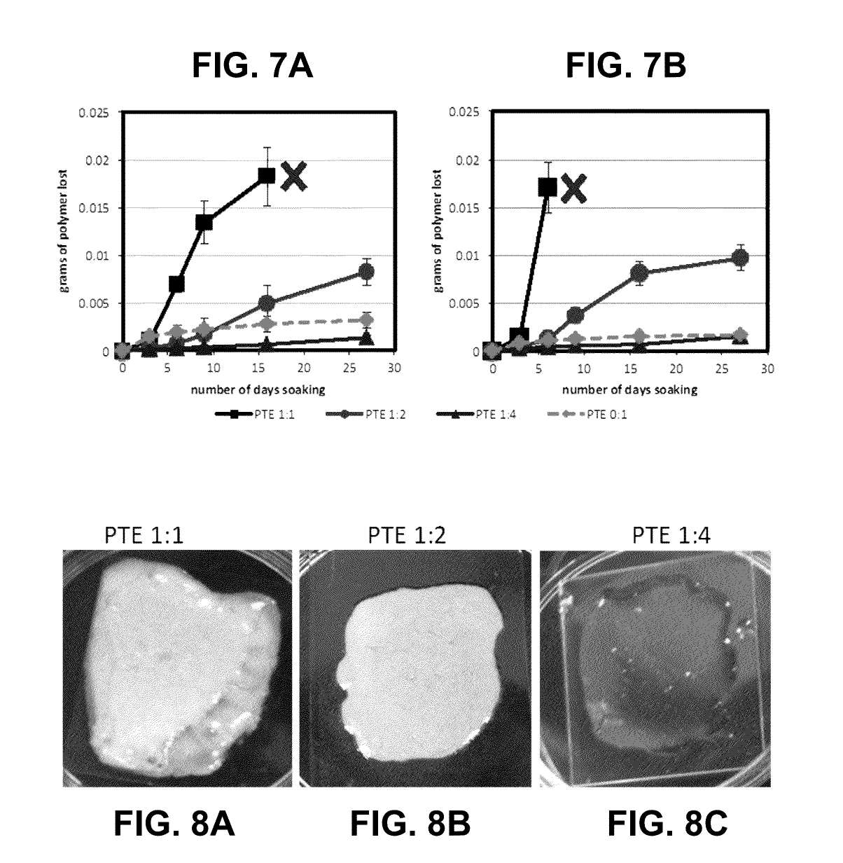

FIGS. 7A and 7B are graphs illustrating dissolution properties of PTE films. The dissolution properties of PTE's were studied by soaking bulk PTE films in 0.1 M sodium phosphate buffer at room temperature. The effect of pH on film degradation was examined by utilizing sodium phosphate buffer at pH 5.7, pH 7.4 and pH 8. As shown by the polymer weight loss at pH 7.4 (FIG. 7A) the rate of polymer weight loss decreased with decreasing 4PA content. Although the sample with no 4PA initially degraded more rapidly than PTE 1:2 and PTE 1:4 samples, it is believed that this degradation is a physical change in the polymer, rather than a chemical degradation.

Polymer weight loss was studied at pH 5.7, pH 7.4 and pH 8. For both PTE 1:1 and PTE 1:2, dissolution occurred more rapidly at higher pH, consistent with the increased susceptibility of anhydride bonds to hydrolysis at higher pH. The dissolution rate and water absorption profile of PTE 1:4 appeared less significantly related to pH (FIG. 7B).

FIGS. 8A-8C are images depicting the appearance of the PTE films of FIGS. 7A and 7B upon wetting. Over a 16-day period the films lost less than 1 mg of dry weight and absorbed less than 1 mg of water. Additionally, the appearance of PTE 1:4 remained unchanged after 16 days, whereas PTE 1:1 and PTE 1:2 became white, sticky and in some instances gel like after extended incubation.

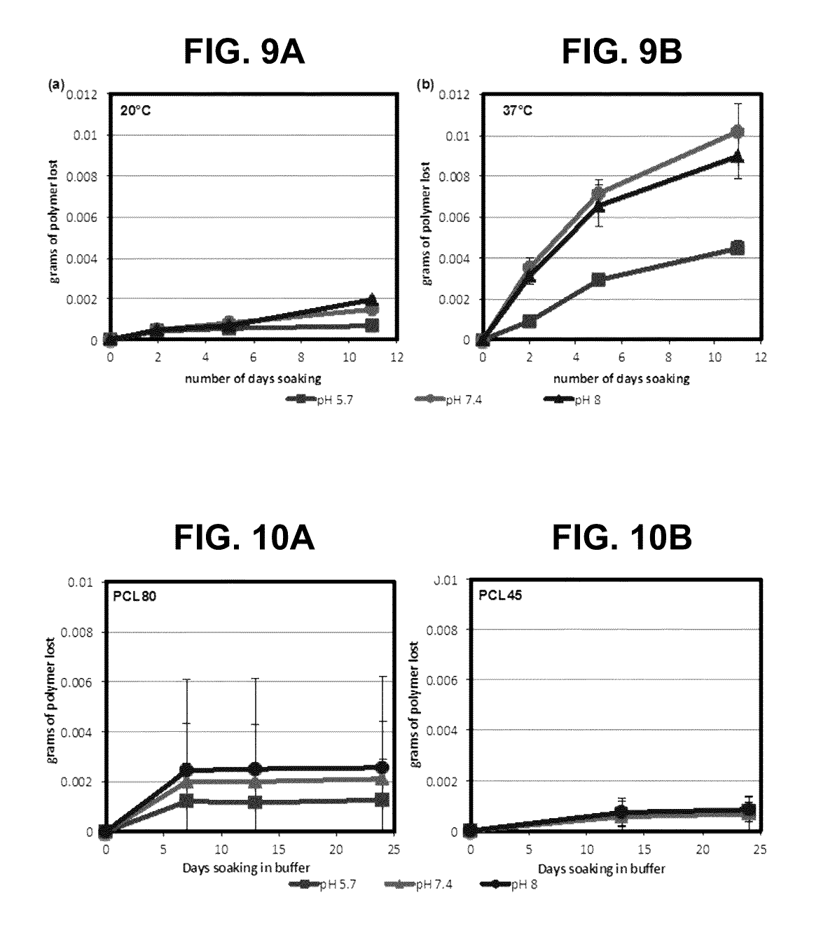

As shown in FIGS. 9A and 9B, based on the promising characteristics of PTE 1:4, the dissolution of PTE 1:4 was studied at 37° C. to understand material changes at physiological temperature. FIG. 9A is a graph illustrating polymer weight loss of PTE 1:4 at room temperature and FIG. 9B is a graph illustrating polymer weight loss of PTE 1:4 at 37° C. As shown, the dissolution and water uptake of PTE 1:4 were accelerated by 10-fold at 37° C. as compared to room temperature. Additionally, further work completed in the Rogers lab showed that PTE 1:4 was able to protect a patterned Mg resistor for 5 days; however upon addition of three SiO₂/SiN layers, the lifetime was extended to 27 days. Based on this data, it can be concluded that PTE serves as a lead candidate material for the substrate and/or encapsulation material of the medical device.

PCL is a relatively simple polyester that is available commercially in many different molecular weight formulations, from which we selected Mn 14,000 (PCL14), Mn 45,000 (PCL45) and Mn 80,000 (PCL80) to cover a wide range of properties. Solvent cast films of PCL14 did not have any structural integrity and demonstrated significant cracking and flaking, and was not evaluated further. In PCL80 formed robust and controllable thin films via solvent casting and spin coating. PCL film thickness was easily controlled by spin coating speed.

The dissolution properties of PCL were studied via a polymer weight loss test in 0.1 M sodium phosphate buffer, as described above. As shown in FIGS. 10A and 10B, the degradation of both types of PCL is slow, and occurs with low water uptake. Specifically, PCL80 lost approximately 2 mg of polymer (equating to 2% of the original weight) in 13 days, while PCL45 lost less than 1 mg of polymer (equating to less than 1% of the original weight) in 24 days. There was no apparent effect of buffer pH, as expected since the degradable ester bonds in PCL require more extremes of pH to affect hydrolysis. Importantly, the structural integrity of the PCL films remained intact throughout the dissolution test with some flaking of the material visible on the polymer surface. Similarly, both PCL45 and PCL80 showed low water uptake of less than 0.025 mg in 24 days, equating to less than 0.1% water uptake. Thus, PCL has emerged as one of the lead candidate materials for evaluation in the context of transient electronics.

Of the four classes of POE materials, only those from Classes II and IV met the criteria for experimental evaluation. POE's are not available commercially, and thus required synthesizing. The monomer for the type IV POE's, adiketeneacetal called “DETOSU” (1), is not commercially available, and was synthesized by the rearrangement of diallylpentaerythritol(2) via KOtBu in ethylene diamine, as shown below:

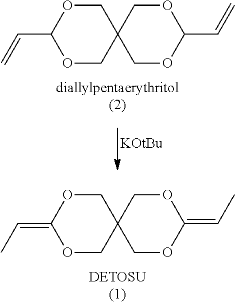

Melt polymerization of DETOSU with trans-cyclohexanedimethanol (tCHD) and 1,6 hexanediol (HD) in the ratio of DETOSU:tCHD:HD (100:50:50) gave the target material, thin films of which were produced by solvent casting.

Type IV POE's provide an orthogonally reactive option for substrate and encapsulation materials by virtue of their sensitivity to lower pH versus the high pH sensitivity observed for PTE, PCL and other ester-containing polymers. While this class remains of interest, the exothermic acid-catalysed polymerization raised significant concerns regarding future manufacturing at scale. Despite literature claims that the exotherms are manageable, we elected to temporarily de-prioritize this class given the promising results obtained with other systems (e.g. PCL, PTE). Future work on this class would involve detailed investigations into a wide range of linker molecules and linker ratios, and alternative, less exothermic methods of polymerization.

PGS is an elastomeric polyester formed through polycondensation of glycerol and sebacic acid at a mole ratio of 1:1 or 2:3. PGS films were fabricated via drop casting and spin casting of hot PGS prepolymer, followed by curing at 120° C. under vacuum. The polycondensation of glycerol and sebacic acid to form PGS (1:1) elastomer is shown below:

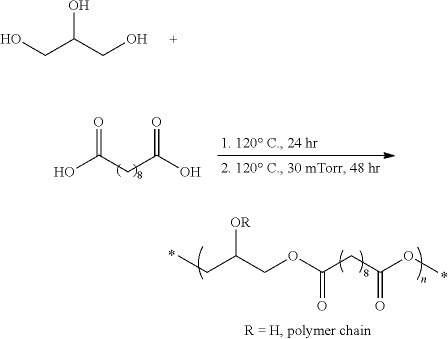

Film thickness has proven difficult to control due to frequent irregularities in curing, which in turn cause a dewetting effect in the material. Thicker cast films have been more consistent from batch to batch.

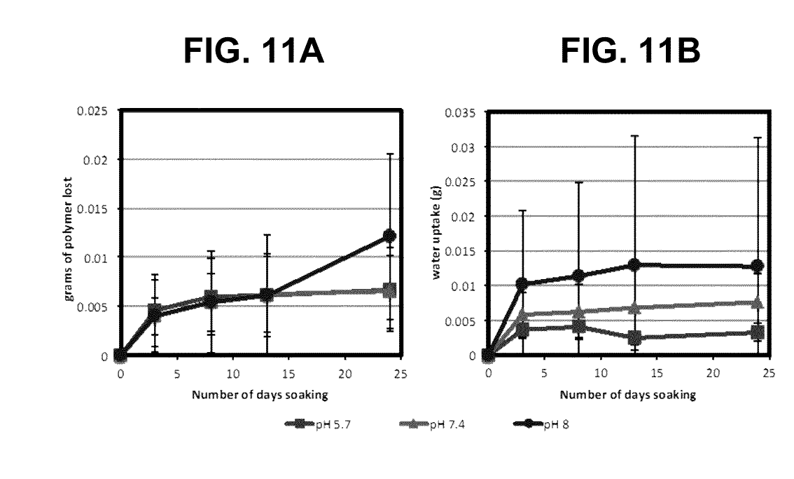

The dissolution properties of PGS (1:1) were studied via a polymer weight loss and water uptake study as described above. FIGS. 11A and 11B are graphs illustrating dry polymer eight loss and water uptake for PGS (1:1), respectively. FIG. 11A shows the weight loss of PGS (1:1) over 24 days. PGS (1:1) lost 6-10 mg of weight over the course of 24 days (0.46 mg/day), with no apparent effect from buffer pH, analogous to the results obtained for PCL films. As shown in FIG. 11B, PGS (1:1) showed a significantly higher water uptake than PCL and PTE's, with 10 mg of water uptake after only 3 days.

Despite the high hydrophobicity of PGS as indicated by the high contact angle (see Table 1), the dissolution data show a clear propensity for water uptake. This phenomenon may be attributable to the high porosity of PGS, a result of its highly branched polymer structure. The challenges in achieving reproducible thin films in combination with the relatively high water uptake rates led to the decision to de-prioritize the PGS class as a candidate for further evaluation.

Accordingly, three candidate materials for the substrate and/or encapsulation layer were identified. The two leading candidates, PCL and PTE, were further tested with a circuit of a medical device previously described herein. Additional characterization of these materials will continue, however, in order to elucidate more detailed physical properties and behaviors of the encapsulating materials. Further optimization of PCL and PTE will continue in order to extend the physical lifetime of the materials. Solvent casting and spin coating were the primary methods for producing thin films during Phase I. However, we recognize that robust product development requires highly reproducible films in terms of thickness, crystallinity and residual solvents, and solvent cast films are prone to physical defects, which may skew dissolution and mechanical properties. Additionally, fully removing solvent from the polymer can be challenging, and residual solvent can act as a plasticizer and alter the polymer's mechanical properties. Finally, solvent cast films are typically amorphous as opposed to heat processed films, in which crystallinity is typically more controllable.

The thermal stability of substrate films influences the processes selected for the assembly of electronics. The thermoplastic characteristics of PCL, and its moderately low melting point (59-64° C.), enable the facile melt processing of PCL, but limit the high temperature electronics deposition processes. Low temperature deposition of electronics, such as transfer printing, will be required for this material. In contrast, the PTE materials, resistant to at least 150° C., will be stable in higher temperature thermal/E-beam metal deposition techniques. A more thorough evaluation of deposition and circuit fabrication techniques will be undertaken during Phase II.

The long-term stability of all materials must be improved for their use as substrates and encapsulants in implantable devices. This can be achieved via copolymerization, blending of polymers and material layering. Advanced polymer processing, such as hot pressing to form more crystalline films, may also improve material stability.

As previously described, one or more components of the circuit of the medical device may include materials and/or have specific dimensions resulting in predictable and controllable resorption rates, such that the medical device 202 may cease to function and completely dissipate within a medically relevant timescale (e.g., after completion of treatment). For example, one or more of the components of the circuit include a material selected from the group consisting of Mg, Mg alloys, MgO, Zn, W, Fe, Mo, Si, SiO₂, and combinations thereof.

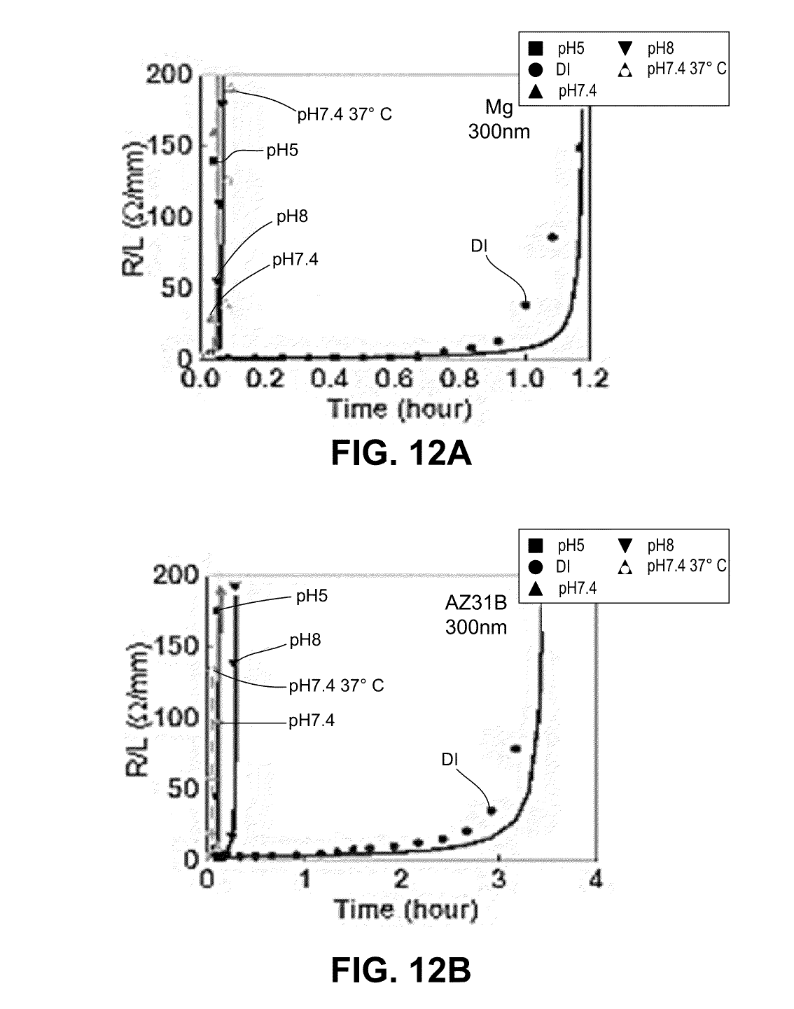

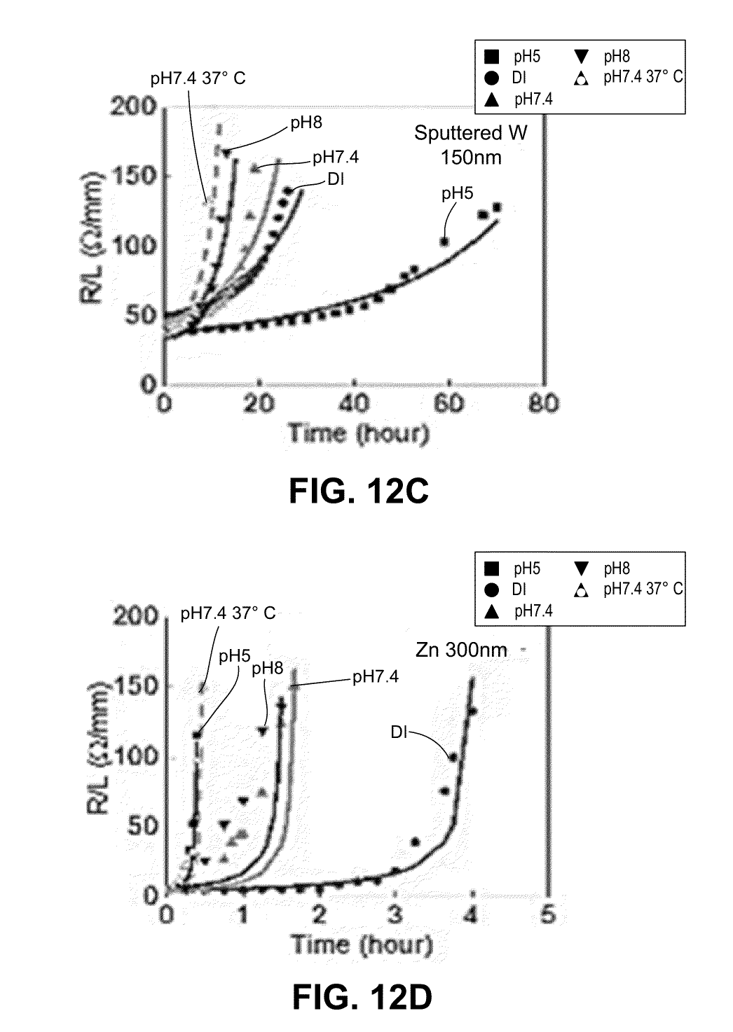

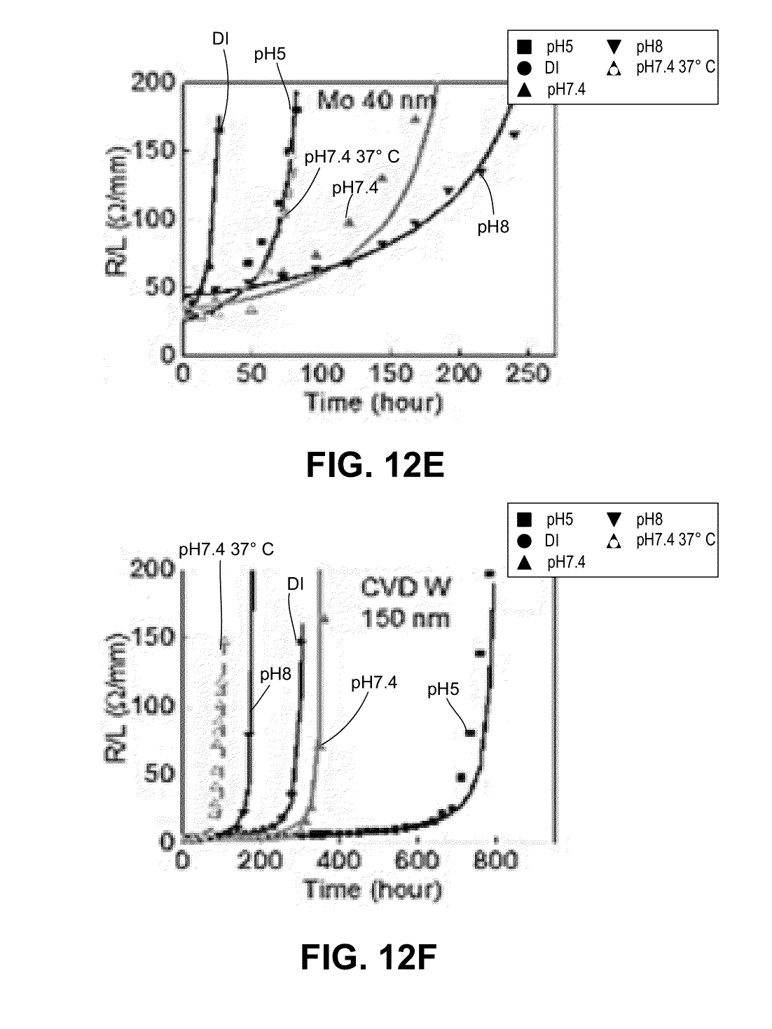

FIGS. 12A-12F are graphs illustrating resistance changes during the dissolution of different exemplary bioresorbable metals for use as one or more components in the circuit of a transient medical device consistent with the present disclosure. As shown, the degradation profiles of different metal materials are under evaluation with the intent to identify metals that could be implemented in transient electronic systems. Six metals that are degradable, bio-resorbable and compatible with silicon devices were evaluated in dissolution studies: Mg, Mg alloy AZ31B (with 3 wt % aluminum (Al), and 1 wt % Zn), W, Zn, and molybdenum (Mo). The dissolution behavior of these metals, as measured by resistance, was investigated in de-ionized (DI) water and simulated bio-fluid (Hank's solution) over a pH range of 5-8. Metal thin films of 150 nm or 300 nm were fabricated by either E-beam evaporation (Fe) or magnetron sputtering techniques. When compared to changes in film thickness over the same time period (not shown), it is evident that resistance changes much faster than thickness, indicating that resistance may be a better indicator of degradation in the context of device function. When considering metals as substrates however, thickness may be a more relevant measurement.

Based on the dissolution results in FIGS. 12A-12F, the following points were concluded: Mg, Mg alloy and Zn dissolve at much faster rates versus Fe and W; Mg, Mg alloy and Zn dissolve at faster rates in HBSS versus DI water; dissolution rates are nearly pH independent; and sputtered W degrades slower in DI water and pH 5 Hank's solution due to its acidic dissolution products. As such, the rates of dissolution appear faster for Mg, Mg alloy and Zn than that for Mo and W, and salt solutions significantly enhance the degradation rates compared to DI water, except that the rates for Mo and W in pH 5 solution. Dissolution rates of W can also be modified by a factor of ten through different deposition methods.

Surface morphology and metal microstructure were also studied as a function of dissolution time to provide insight into dissolution mechanisms (data not shown). Optical microscopy and SEM data indicate that the dissolution behavior is more uniform for Mg, AZ31B and W compared to Fe and Zn. A combination of SEM, X-ray diffraction (XRD) and X-ray photoelectron spectroscopy (XPS) analysis indicate the presence of surface oxide during the course of dissolution. Magnesium hydroxide around 10-20 nm was detected on the surface of Mg and Mg alloy, and both the Mg metal and MgO almost completely disappear at the later stage. Tungsten hydroxide and ZnO were observed on W and Zn respectively, and while the oxides did not dissolve completely, the metal phases gradually disappeared.

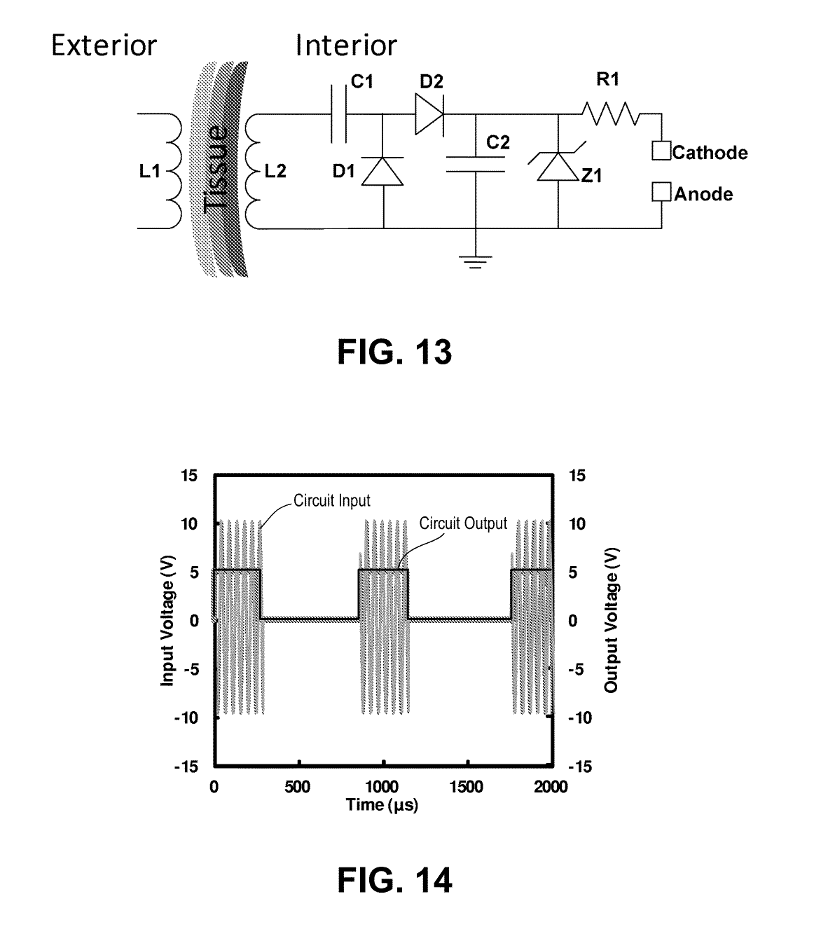

FIG. 13 illustrates one embodiment of a circuit of the transient medical device of the system of FIG. 1 consistent with the present disclosure. As shown, the implantable circuit for providing electrical stimulation to management pain includes inductive coil L2 configured to wirelessly communicate with an inductive coil L1 of the external controller, a rectification circuit (formed by capacitors C1 and C2, diodes D1 and D2), PIN diodes (Z1), current limiting resistors (R1), cathodes, and an anode. The coil L1 of the external controller can operate in two different modes, in which a constant sinusoidal wave or a modulated sinusoidal wave can be generated, resulting in different outputs from the implanted circuit. The implanted coil L2 communicates with external coil L1 through the skin and/or tissue via resonant inductive coupling, for example.

C1, C2, D1, and D2 combine to form a voltage doubler that changes the input alternating current (AC) to a direct current (DC) output voltage whose amplitude is twice as large as the input voltage. The PIN diode Z1 is configured to regulate the DC voltage to be approximately 5.1 V, while resistor R1 limits the output current to a level tolerable by human tissue. In turn, the electrodes (cathodes and anode) are configured to generate and deliver an electric field having a frequency in the range of 6 to 14 MHz. In one embodiment, the frequency of the electric field is 6.78 MHz, which is comparable to the ISM standard for implantable medical devices while maintaining a large quality factor for inductive coupling through inhomogeneous human tissue. In addition, the theoretical skin penetration depth of the electromagnetic field at 6.78 MHz is approximately 0.97 m, thus ensuring consistent inductive coupling to the implanted circuits regardless of its placement locale within the patient.

While the size of the rectification circuit is fixed and expected to be 5×5 mm, the dimension of coils and electrodes vary according to the specific application. Larger coil dimensions provide higher efficiency in capturing the external electromagnetic field and longer working distances, but the external coil with comparable size to the implanted coil can be used to optimize overall dimension of the circuit. In one embodiment, the electrodes are configured to deliver between 1 to 10 mA of current in monophasic square-wave pulses having durations between 10 to 200 is to provide between 10 to 2000 nC of charge to one or more nerve fibers. The pulses may be delivered to the one or more nerve fibers have a frequency in the range of 40 to 200 Hz. The electrodes are configured to deliver a variety of different stimulation patterns (e.g., different electrical fields) based on wireless input from the external controller. For example, the external controller may operate in a variety of different modes, each mode resulting in the delivery of a different stimulation pattern from the electrodes. Accordingly, the circuit is configured to allow adjustment, or tuning, of the stimulation patterns on a patient-by-patient basis for frequency, amplitude and duration so as to inhibit the transmission of pain signals along the nerve fibers, thereby providing pain relief. The external controller electronics and coil L1 may include standard, non-transient technologies, ultimately assembled in compact enclosures with user friendly interface. For the purposes of present disclosure, off-the-shelf function generators, amplifiers, coils and associated control equipment were used.

FIG. 14 is a graph illustrating exemplary circuit input and circuit output for peripheral nerve stimulation. It has been demonstrated that electrical nerve stimulation with a median pulse width of 300 us and a current level of 2.5 mA can generate effective parasthesias. Accordingly, the electrodes are configured to deliver monophasic, sinusoidal capacitively-coupled output pulses to the one or more nerve fibers based on wirelessly received input from the controller.

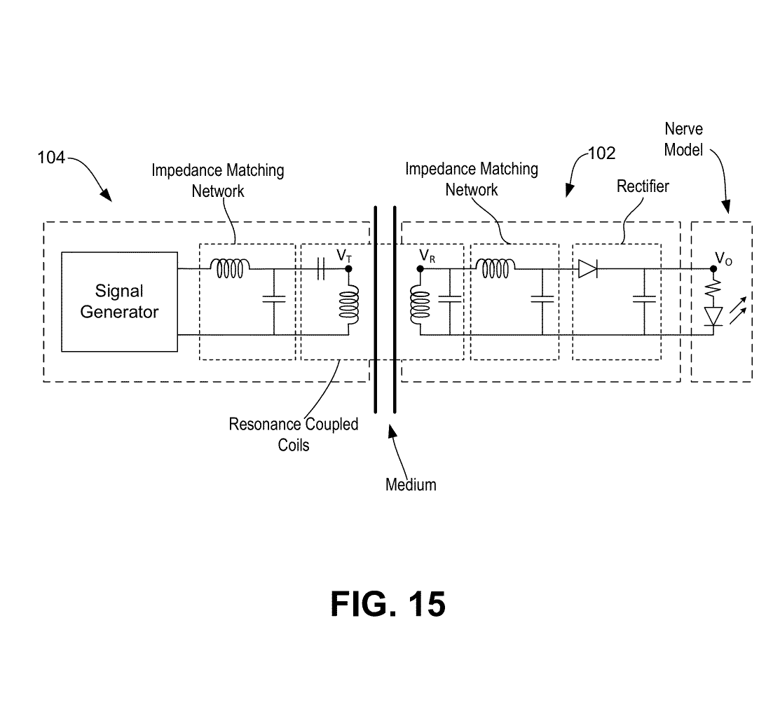

FIG. 15 illustrates another embodiment of a circuit of the external controller and transient medical device of the system of FIG. 1 consistent with the present disclosure. The circuit of FIG. 15 is a demonstration circuit designed to deliver 200 μsec-long monophasic pulses of 5 mA of current to a fixed 500Ω resistive load with a frequency of 100 Hz. A circuit capable of delivering stimulation with these parameters can stimulate the sciatic nerve of a rat and will provide a basis for testing of the entire transient stimulation system in an in vivo experiment.

The proof-of-concept nerve stimulator system was designed to receive wireless power via resonant inductive coupling from an external controller 104 in the form of a PCB-based transmitter operating at a transmission frequency within the 13.56 MHz ISM band. Planar rectangular spiral inductors were used as transmitting and receiving antennae; the transmitter was designed to be positioned on the exterior surface of the tissue while the receiver was designed to be implanted 10 mm below the skin surface.

The coupling frequency of the two spiral coils was chosen based on the allowable size of the implantable receiver coil (10 mm outer diameter), the separation between the transmitter and receivers, and ISM regulations for radiation absorption in tissue. Resonant coupling between the primary and secondary coils can greatly improve the power transmission efficiency for near-field inductively coupled systems. Capacitors are added in series or parallel with each coil to create two resonant tanks with equivalent resonant frequencies. The receiver circuit rectifies the coupled power using a half-wave rectifier and delivers it to a 500Ω resistive load. An indicator LED placed in series with the resistive load provides visual confirmation of the power delivered to the load.

Initial designs for the controller (also referred to herein as “transmitter”) and medical device (also referred to herein as “receiver”) coils were chosen based on existing non-transient biomedical implant power transmission circuits. The inductance of the primary and secondary coils, as well as the necessary resonant capacitance required on both the primary and secondary, were selected to maximize the power transmission through saline solution (a simplified lab-based model for biological tissue).

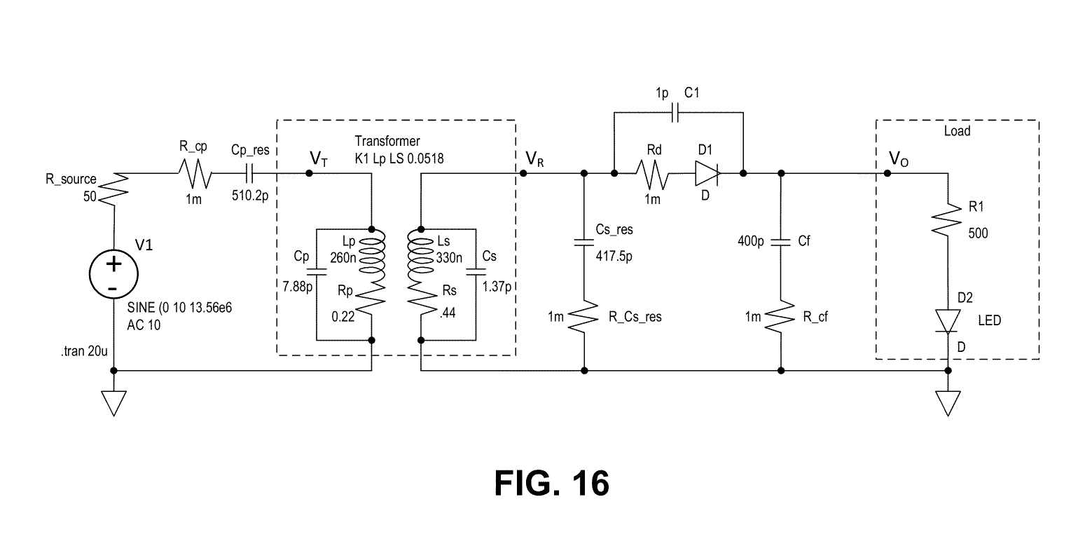

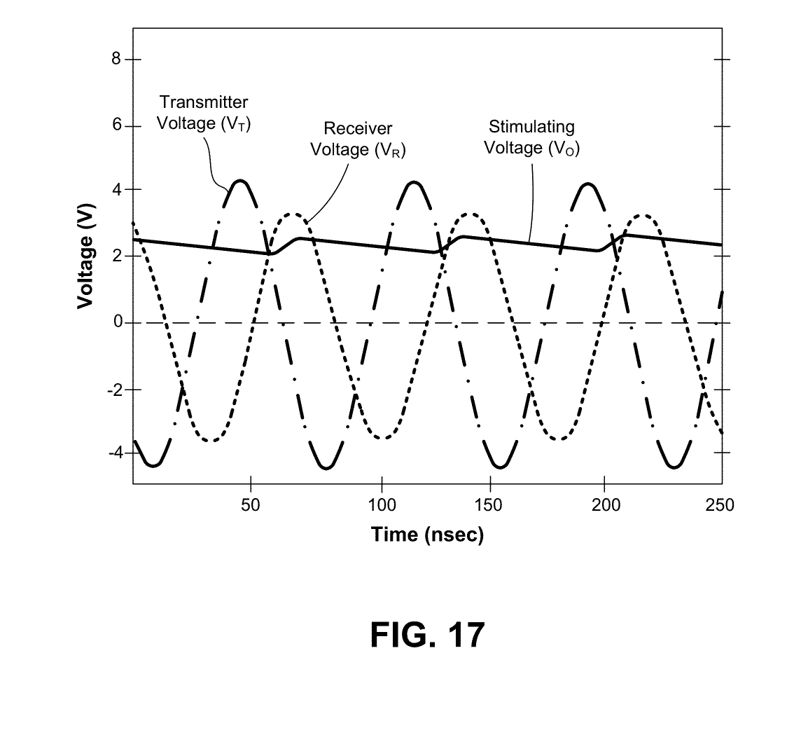

FIG. 16 illustrates another embodiment of a circuit of the external controller and transient medical device of the system of FIG. 1 consistent with the present disclosure. The circuit of FIG. 16 was simulated using circuit simulation software, specifically LTSPICE IV, a SPICE simulator, commercially available from Linear Technology Corporation. The circuit was simulated using detailed models of the parasitic resistance and capacitance of each component. FIG. 17 is a graph illustrating different voltages observed in the circuit of FIG. 16 upon simulation of the circuit. The stimulating voltage has an average value of 2.5 V, which corresponds to an output current of 5 mA through the load. Based on the simulations, the predicted capacitance values required to resonate the secondary coil and to smooth the output to provide monophasic stimulation represent a 50-fold increase in energy storage capacity over the state-of-the-art capacitors currently developed.

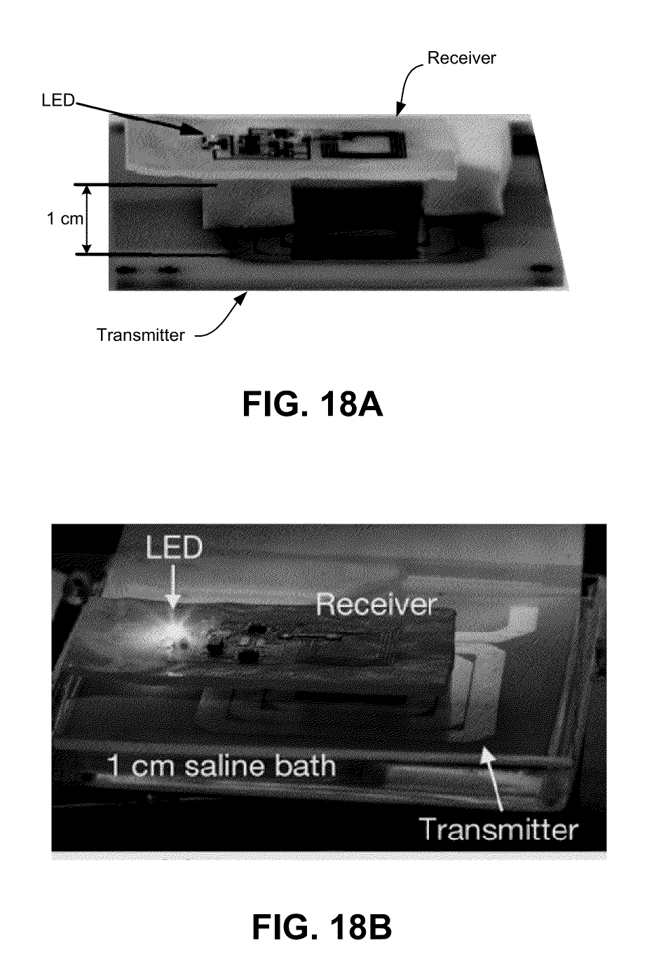

FIGS. 18A and 18B are perspective views of an exemplary external controller (e.g., transmitter) wirelessly communicating with an exemplary transient medical device (e.g., receiver) through different mediums (air in FIG. 18A and saline solution in FIG. 18B). The functionality of a transient medical device of the present disclosure was demonstrated by transmitting wireless power to a medical device (e.g., receiver) circuit and illuminating an indicator LED with 2 mA of current. Wireless function was established with two different experimental configurations: a first configuration with 1 cm of air separating the transmitter and receiver (shown in FIG. 18A, and second configuration with 1 cm of saline solution separating the two circuits (shown in FIG. 18B). The indicator LED, which was connected in series with a 500Ω output resistor, was illuminated for both conditions, confirming that sufficient voltage was received by the stimulator circuit in both cases.

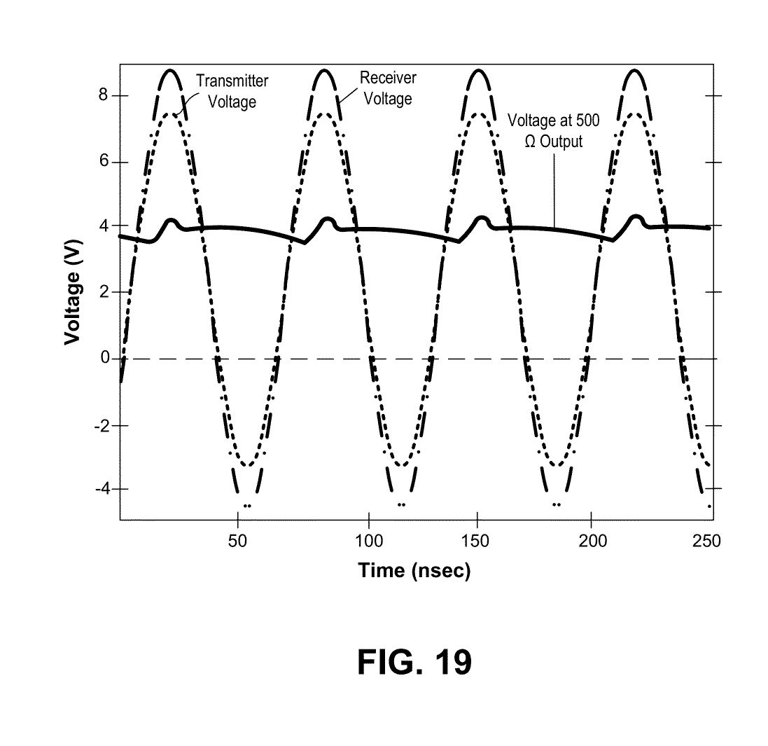

FIG. 19 is a graph illustrating different voltages observed during operation of the systems of FIGS. 18A and 18B. The transmitter and receiver coils were resonated at 13.56 MHz to boost the voltage at the receiver unit. A signal generator provided a 5 V peak-to-peak sinusoidal signal to the transmitter PCB. This waveform was generated in “burst mode” with a 10 msec period and 200 μsec pulse width to satisfy the requirements of the nerve stimulator. A rectified voltage of 1 V was provided to the output resistor, as shown in FIG. 19, thereby illuminating the LED with 2 mA of current. These circuit demonstrations validate our overall circuit design and wireless power transmission system.

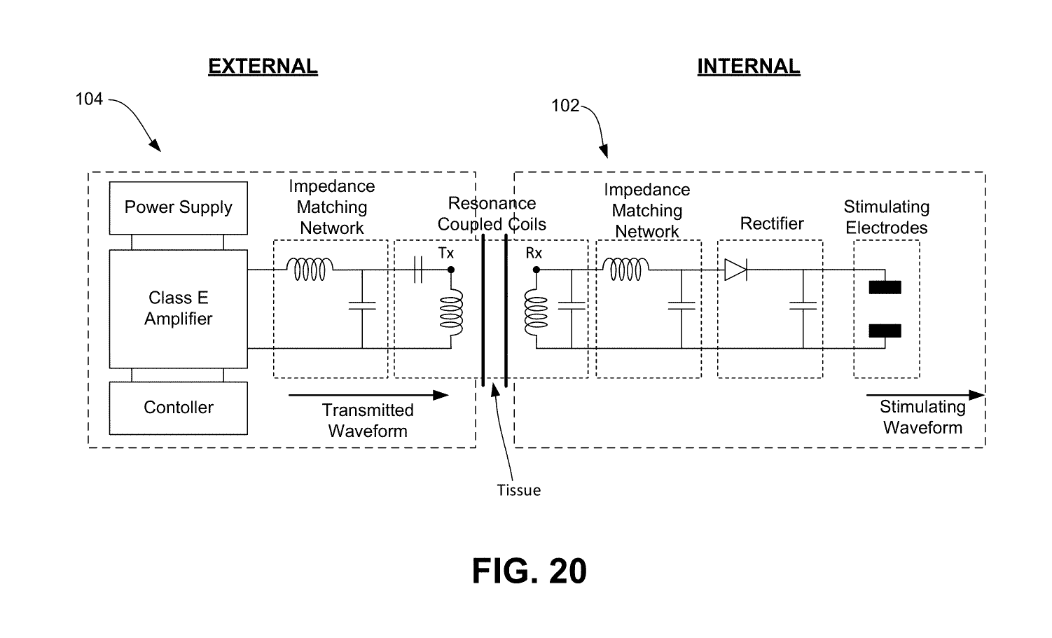

FIG. 20 illustrates another embodiment of a circuit of the external controller and transient medical device of the system of FIG. 1 consistent with the present disclosure. A battery-powered class-E amplifier will generate a 13.56 MHz sinusoidal wave form whose amplitude is modulated by a controller. The peak voltage of the transmitted waveform will be limited to a safe value to prevent excessive power from being delivered to the nerve tissue. The sinusoidal waveform generated by the amplifier will be wirelessly transmitted in pulses whose width is set by the control circuitry. The transmitted waveform will be coupled through tissue using resonant inductive coupling to maximize the power transferred to the implanted circuit. Impedance matching circuits on the transmitter and receiver will be used to tune the load impedance to maximize the power transfer from the amplifier to the transmitter antenna and from the receiver antenna to the tissue. The impedance matching network on the transmitter will be used to match the output impedance of the class-E amplifier to the impedance of the resonant transmitter coil. Similarly, on the receiver side, the impedance matching network will be used to match to impedance of the receiver coil to that of the tissue (nominally 500Ω).

The matching networks shown in FIG. 20 are L-match networks that are used when the load impedance is larger than the source impedance. The source and load impedance on the transmitter and receiver sides will both be measured experimentally to optimize the impedance match structure and component values to maximize the power-transfer efficiency. A half-wave rectifier will convert the ac voltage waveform to dc to drive the nerve tissue with monophasic square wave pulses. A filter capacitor with sufficient capacitance will be used to smooth the voltage to within a 10% peak-to-peak voltage ripple on the output. Simulations of this circuit predict that capacitance values between 200 and 500 pF will be needed to smooth the output voltage of the circuit and to resonate the secondary coil at 13.56 MHz.

Monophasic stimulation of nerve tissue has been shown to be safe and effective when implemented at charge densities below 0.2 μC/mm² per pulse. The circuit of the present device is designed to operate below this safety threshold to mitigate tissue damage associated with Faradaic charge delivery and tissue electrolysis.

The circuit will deliver 1-10 mA of current in monophasic square-wave pulses with durations of 10-200 μsec to peripheral nerve tissue. This charge will be delivered to the tissue over short pulses with a frequency between 40 and 200 Hz to stimulate paresthesia in the patient. These requirements have been demonstrated to be effective in mediating pain both in animal and human studies. Monophasic stimulation that delivers charge at a density of 0.2 μC/mm² per pulse resulted in no tissue damage in previous studies. The largest charge per phase that our stimulator will be able to deliver will be 2 μC/phase. Given a 10 mm² electrode area, the charge density per phase would be at most 20 μC/mm²/phase, which is well within the levels of safe stimulation.

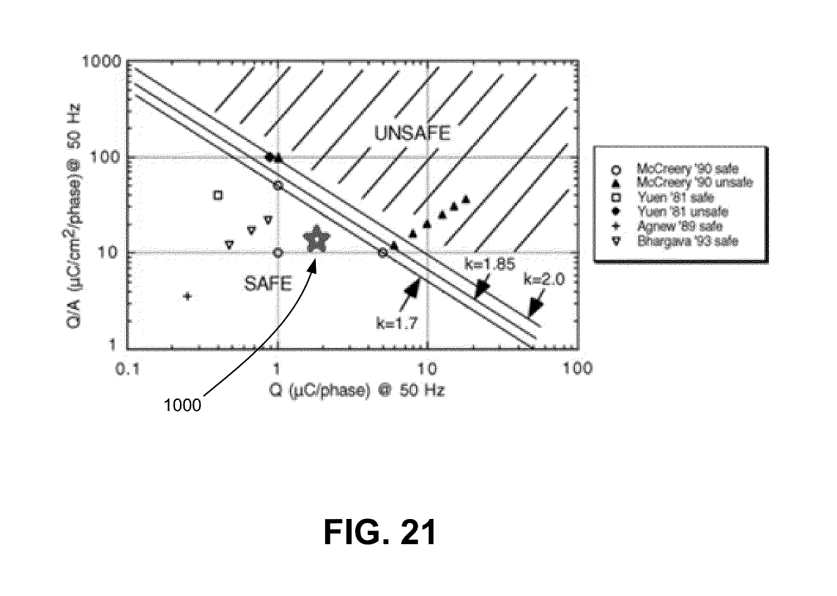

FIG. 21 is a graph illustrating a range of tolerant stimulation levels configured to be delivered by the circuitry of the transient medical device of FIG. 20. The highest allowable stimulation provided by the circuit of FIG. 20, as indicated by arrow 1000, is still within the safety limits demonstrated in previous experiments. The transmitted pulse width, amplitude, and frequency will be set by external control circuitry on the transmitter PCB. The controller will be able to adjust the transmitted waveform parameters over the following ranges: pulse widths between 10-200 μsec, transmitted voltage amplitudes between 5 and 15 V, pulse frequencies between 40-200 Hz.

In some embodiments, the implantable transient medical device of the present invention is configured to operate without direct feedback, such that unidirectional power for stimulation will be the only wireless signal transmitted in the system. Accordingly, the output voltage from the medical device can be regulated by limiting the peak voltage delivered from the external controller. Simulations and lab tests will be done to determine at which level to set this peak threshold voltage considering the range of possible coupling factors between the primary and secondary coils. An additional level of control can be added, if deemed necessary after this testing, which would limit the peak voltage of the stimulating waveform using a Zener diode. A Zener diode would serve as over-voltage protection and would provide a fixed voltage output for coupling factors and transmitted voltages greater than chosen values. The complexity of the control and regulation circuits will be determined in part by the patient condition that our stimulator serves to treat and the precise location in the body in which we intend to implant the device. Additional levels of control can be added to ensure tighter regulation of the electrical stimulus provided by our implant.

Accordingly, an implanted transient stimulator circuit consistent with the present invention may have a total device surface area of no more than 5 cm² and will deliver 1-10 mA of current in monophasic square-wave pulses with durations of 10-200 μsec to provide between 10 and 2000 nC of charge to peripheral nerve tissue. These charge pulses will be delivered to the tissue with a frequency between 40 and 200 Hz to stimulate continuous paresthesia within the nervous system to mask sensations of pain. These stimulation requirements have been demonstrated to be effective in mediating pain both in animal and human studies. The stimulator will be wirelessly powered by an external power supply circuit. The frequency and peak current amplitude of the stimulus pulses will be tunable based on the transmitted voltage waveform from the external circuit.

A battery-powered transmitter circuit utilizing non-transient integrated circuit components may be positioned on the exterior surface of the tissue (positioned directly over the implant) and provide wireless power to the implant via near-field inductive coupling at a desired frequency, such as, for example, 13.56 MHz. Resonant inductive coupling between an external coil and an implanted coil will be used to deliver power to the stimulator circuit. The transmitter and receiver coils will each be connected to capacitors to form resonant tanks that oscillate at 13.56 MHz to maximize the transfer of power through the tissue. The external controller and implanted medical device may be loosely coupled through tissue at a nominal distance of 10 mm, for example. The external controller is configured to wirelessly deliver unidirectional electrical power from a class E amplifier to the implanted medical device. The transmitted power will be limited such that the power delivered to the target tissue does not exceed safety thresholds.

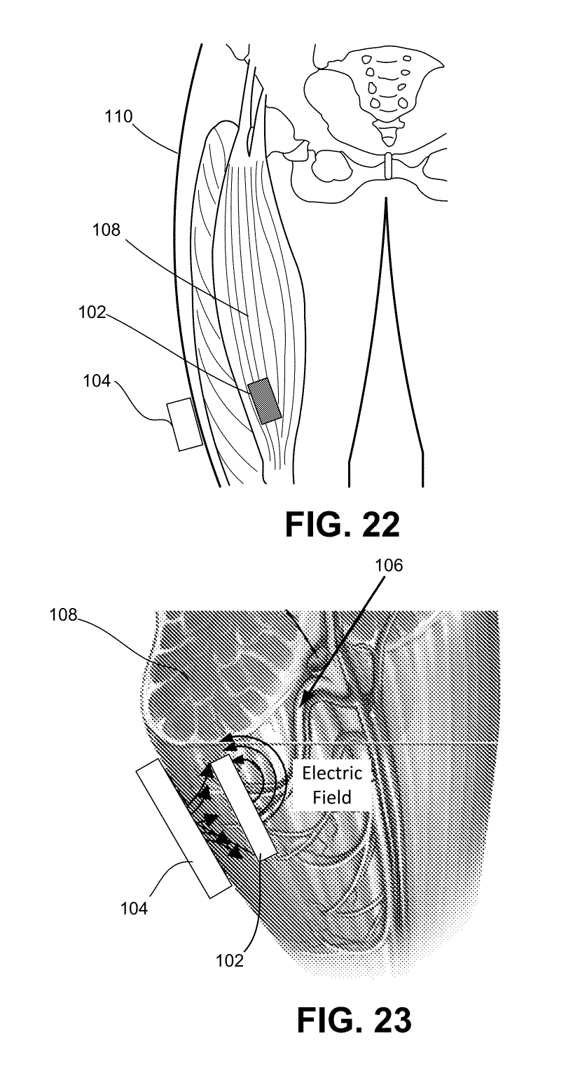

FIG. 22 is a sectional anterior view of a portion of a patient's torso, illustrating the implantation of a transient medical device 102 consistent with the present disclosure adjacent to the rectus femoris muscle of the leg. FIG. 23 is an enlarged view, partly in section, of the rectus femoris muscle 108 including a bundle of peripheral nerves 106 targeted with the electrical field generated and delivered from the transient medical device 102.

As shown, the medical device 102 may be implanted subcutaneously at or in close proximity to a trauma site, such as a bundle of nerve fibers 106 in the rectus femoris 108 of a patient's leg. The controller 104 is disposed externally to the patient's leg in close proximity to the medical device 102. The medical device 102 may be immobilized at the time of transplantation by way of bioresorbable fixtures, such as sutures or staples (not shown). In one embodiment, the fixtures are configured to degrade at the same rate as the implanted medical device. In another embodiment, the fixtures may provide temporary immobilization until the medical device is fixed within the implant site via immunologically-driven encapsulation by fibrous extracellular matrix material. In another embodiment, the circuit and substrate of the medical device 102 may be sufficiently flexible such that the medical device may be configured to physically conform to the implant site and/or target nerves, thus precluding the requirement for immobilization.

Nerve stimulation to relieve pain is achieved by wirelessly transmitting high frequency signals from the external controller 104 to the medical device 102, via inductive resonance coupling, for example. Upon receiving high frequency signals, a current flows between the electrodes of the circuit of the medical device 102, wherein the electrodes are configured to deliver electrical energy to the one or more nerve fibers to stimulate paresthesia, thereby masking associated pain. In one embodiment, the electrodes are configured to generate an electric field that penetrates surrounding tissue containing the affected sensory or peripheral nerves. The electrodes are configured to deliver a variety of different stimulation patterns based on wireless input from the external controller. For example, the external controller 104 may operate in a variety of different modes, each mode resulting in the delivery of a different stimulation pattern from the electrodes. Accordingly, the system allows the tuning of stimulation patterns on a patient-by-patient basis for frequency, amplitude and duration so as to inhibit the transmission of pain signals along the nerve fibers, thereby providing pain relief.

The wireless capabilities of the external controller 104 and implanted device 102 allow improved treatment. For example, as shown, the external controller 104 need only be placed adjacent to the implanted device 102 so as to provide power to and stimulation from the device 102, without requiring a directly wired connections. For example, some implantable devices must be directly connected to an external power source or controller in order to function, wherein, in addition to being inconvenient to a patient, the wire connecting the external power source or controller and the device must be constantly cleaned and monitored to avoid infection. The ability to wirelessly control of the medical device 102 of the present invention overcomes the drawbacks associated with wired connections, thus improving patient treatment and compliance.

As previously described herein, the substrate and one or more of the electronic components of the circuit of the medical device 102 are bioresorbable and biocompatible. In one embodiment, the substrate and most, if not all, of the components of the circuit have specific dimensions or geometries resulting in predictable and controllable resorption rates, such that the medical device 102 may cease to function and completely dissipate within a medically relevant timescale (e.g., after completion of treatment). Accordingly, once the functional phase of the device 102 is terminated, the remnants of the implanted device 102 may be resorbed naturally over a much longer time period without requiring surgery on the patient's leg to remove the device 102.

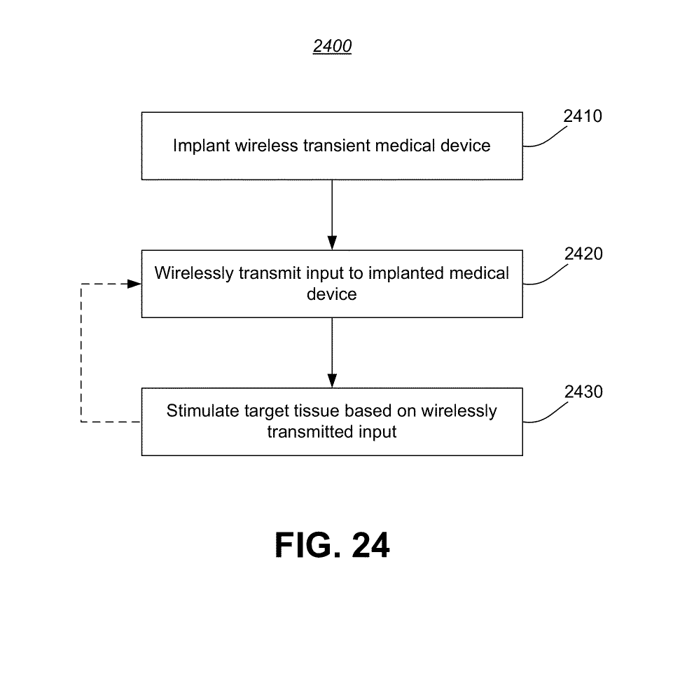

FIG. 24 is a flow diagram illustrating one embodiment of a method 2400 for stimulating a target tissue within a body of a patient. The method includes implanting a medical device with the patient's body (operation 2410). Implantation may occur at or near a site of trauma or the target tissue, such that a stimulatory signal from the medical device will reach and address the relevant target, although direct contact between the electrodes and the target tissue itself is not necessary. The method 2400 further includes wirelessly transmitting input to the implanted medical device from a controller disposed external to the patient's body (operation 2420). The wireless transmission may include transmitting power from the controller to the implantable medical device via resonant inductive coupling. The method 2400 further includes stimulating the target tissue based on the wirelessly transmitted input (operation 2430). In some embodiments, the stimulation may be in the form of an electrical field, wherein, in the event the target tissue is a nerve fiber, the electrical field is configured to stimulate paresthesia within the nerve fiber to mask pain.

While FIG. 24 illustrates method operations according various embodiments, it is to be understood that in any embodiment not all of these operations are necessary. Indeed, it is fully contemplated herein that in other embodiments of the present disclosure, the operations depicted in FIG. 24 may be combined in a manner not specifically shown in any of the drawings, but still fully consistent with the present disclosure. Thus, claims directed to features and/or operations that are not exactly shown in one drawing are deemed within the scope and content of the present disclosure.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

What is claimed is:

1. An implantable medical device for stimulating a target tissue within a body of a patient, the device comprising:
a substrate;
a circuit configured to wirelessly communicate with a controller disposed external to the patient's body and configured to provide stimulation to the target tissue; and
a material surrounding the substrate and the circuit, the material configured to break down within the patient's body.

2. The implantable medical device of claim 1, wherein the circuit comprises electronic components.

3. The implantable medical device of claim 1, wherein the circuit comprises an integrated circuit.

4. The implantable medical device of claim 1, wherein the stimulation provided to the target tissue comprises electrical energy.

5. The implantable medical device of claim 4, wherein the target tissue is selected from the group consisting of heart tissue, brain tissue, muscle tissue, epithelial tissue, nerve tissue, and vascular tissue.

6. The implantable medical device of claim 5, wherein the circuit comprises electrodes configured to deliver the electrical energy to stimulate paresthesia within one or more nerve fibers.

7. The implantable medical device of claim 6, wherein the electrodes are configured to generate an electrical field based on wirelessly received input from the controller.

8. The implantable medical device of claim 7, wherein the generated electrical field has a frequency in the range of 6 to 14 MHz.

9. The implantable medical device of claim 6, wherein the electrodes are configured to deliver between 1 to 10 mA of current in monophasic square-wave pulses having durations between 10 to 200 μs to provide between 10 to 2000 nC of charge to one or more nerve fibers.

10. The implantable medical device of claim 9, wherein the pulses delivered to the one or more nerve fibers have a frequency in the range of 40 to 200 Hz.

11. The implantable medical device of claim 6, wherein the electrodes are configured to deliver monophasic, sinusoidal capacitively-coupled output pulses to the one or more nerve fibers based on wirelessly received input from the controller.

12. The implantable medical device of claim 6, wherein the circuit is configured to adjust one or more properties of the electrical energy based on wirelessly received input from the controller.

13. The implantable medical device of claim 12, wherein the one or more properties are selected from the group consisting of amplitude, pulse width, frequency, duration, and combinations thereof.

14. The implantable medical device of claim 1, wherein the material surrounding the substrate and circuit comprises a bioresorbable material.

15. The implantable medical device of claim 1, wherein the material surrounding the substrate and circuit comprises a biodegradeable material.

16. The implantable medical device of claim 1, wherein the substrate comprises a bioresorbable material.

17. The implantable medical device of claim 1, wherein the substrate comprises a biodegradable material.

18. The implantable medical device of claim 1, wherein one or more components of the circuit are bioresorbable.

19. The implantable medical device of claim 1, wherein one or more components of the circuit are biodegradable.

20. The implantable medical device of claim 1, wherein the substrate comprises a material selected from the group consisting of polyanhydrides, polyortho-esters, polyesters, polyphosphazenes, and combinations thereof.

21. The implantable medical device of claim 1, wherein one or more components of the circuit comprises a material selected from the group consisting of Mg, Mg alloys, MgO, Zn, W, Fe, Si, SiO₂, and combinations thereof.

22. A system for stimulating a target tissue within a body of a patient, the system comprising:
an implantable medical device comprising a substrate, a circuit configured to provide stimulation to the target tissue, and a material surrounding the substrate and the circuit, the material configured to break down within the patient's body; and
a controller configured to be disposed external to the patient's body and wirelessly communicate with the device to provide stimulation to the target tissue when the device is implanted within the patient's body.

23. The system of claim 22, wherein the circuit comprises electronic components.

24. The system of claim 22, wherein the circuit comprises an integrated circuit.

25. The system of claim 22, wherein the stimulation provided to the target tissue comprises electrical energy.

26. The system of claim 25, wherein the target tissue is selected from the group consisting of heart tissue, brain tissue, muscle tissue, epithelial tissue, nerve tissue, and vascular tissue.

27. The system of claim 26, wherein the circuit comprises electrodes configured to deliver the electrical energy to stimulate paresthesia within one or more nerve fibers.

28. The system of claim 27, wherein the electrodes are configured to generate an electrical field based on wirelessly received input from the controller.

29. The system of claim 28, wherein the generated electrical field has a frequency in the range of 6 to 14 MHz.

30. The system of claim 27, wherein the electrodes are configured to deliver between 1 to 10 mA of current in monophasic square-wave pulses having durations between 10 to 200 is to provide between 10 to 2000 nC of charge to one or more nerve fibers.

31. The system of claim 30, wherein the pulses delivered to the one or more nerve fibers have a frequency in the range of 40 to 200 Hz.

32. The system of claim 27, wherein the controller is configured to operate in one or more modes, each mode resulting in the electrodes delivering an associated electrical energy to the one or more nerve fibers, each associated electrical energy having corresponding properties.

33. The system of claim 32, wherein the one or more properties are selected from the group consisting of amplitude, pulse width, and frequency, duration, and combinations thereof.

34. The system of claim 32, wherein the controller is configured to operate in at least a first mode, wherein a constant sinusoidal wave is generated and transmitted to the implantable medical device, and a second mode, wherein a modulated sinusoidal wave is generated and transmitted to the implantable medical device.

35. The system of claim 34, wherein the electrodes are configured to deliver monophasic, sinusoidal capacitively-coupled output pulses to the one or more nerve fibers based on wirelessly received input from the controller.

36. The system of claim 22, wherein the implantable medical device and the controller are configured to wirelessly communicate with one another via resonant inductive coupling.

37. The system of claim 22, wherein the material surrounding the substrate and circuit comprises a bioresorbable material.

38. The system of claim 22, wherein the material surrounding the substrate and circuit comprises a biodegradeable material.

39. The system of claim 22, wherein the substrate comprises a bioresorbable material.

40. The system of claim 22, wherein the substrate comprises a biodegradable material.

41. The system of claim 22, wherein one or more components of the circuit are bioresorbable.

42. The system of claim 22, wherein one or more components of the circuit are biodegradable.

43. A method of stimulating a target tissue within a body of a patient, the method comprising:
implanting a medical device with the patient's body, the device comprising a substrate, a circuit configured to provide stimulation to the target tissue, and a material surrounding the substrate and the circuit, the material configured to break down within the patient's body;
wirelessly transmitting input to the implanted medical device from a controller disposed external to the patient's body; and
stimulating the target tissue based on the wirelessly transmitted input.

44. The method of claim 43, wherein stimulating the target tissue comprises generating and delivering electrical energy to the target tissue.

45. The method of claim 44, wherein the target tissue is selected from the group consisting of heart tissue, brain tissue, muscle tissue, epithelial tissue, nerve tissue, and vascular tissue.

46. The system of claim 45, wherein the electrical energy stimulates paresthesia within one or more nerve fibers.

47. The method of claim 46, wherein the circuit comprises electrodes configured to deliver between 1 to 10 mA of current in monophasic square-wave pulses having durations between 10 to 200 μs to provide between 10 to 2000 nC of charge to the one or more nerve fibers.

48. The method of claim 47, wherein the pulses delivered to the one or more nerve fibers have a frequency between 40 and 200 Hz.

49. The method of claim 48, wherein wirelessly transmitting input comprises transmitting power from the controller to the implantable medical device via resonant inductive coupling.
